(19) United States
(12) Patent Application Publication
(10) Pub. No.: US 2025/0018151 A1
(43) Pub. Date: Jan. 16, 2025

(54) MEDICAL DELIVERY SHEATH ASSEMBLY

(71) Applicant: EDWARDS LIFESCIENCES CORPORATION, Irvine, CA (US)

(72) Inventors: Daniel James Murray, Orange, CA (US); Omar Fawzi Azanki, Coto de Caza, CA (US); Steven Park, Santa Ana, CA (US)

(21) Appl. No.: 18/903,821

(22) Filed: Oct. 1, 2024

Related U.S. Application Data
(63) Continuation of application No. PCT/US2023/017205, filed on Mar. 31, 2023.
(60) Provisional application No. 63/327,034, filed on Apr. 4, 2022.

(51) Int. Cl. A61M 25/00

(52) U.S. Cl. CPC A61M 25/0097; A61M 25/0023; A61M 25/0045; A61M 2025/0024; A61M 2205/0216

(57) ABSTRACT

A medical delivery sheath assembly can comprise a first hub portion comprising a first delivery lumen configured to slidably receive at least a portion of a first medical device, and a second hub portion comprising a second delivery lumen configured to slidably receive at least a portion of a second medical device. A longitudinal axis of the first delivery lumen and a longitudinal axis of the second delivery lumen can form an acute angle where a distal portion of the first hub portion and a distal portion of the second hub portion merge. A joint hub portion can be coupled to the distal portion of the first hub portion and the distal portion of the second hub portion. The joint hub portion can comprise a third delivery lumen configured to slidably receive corresponding portions of each the first medical device and the second medical device.

### CROSS REFERENCE TO RELATED APPLICATIONS

This application is a continuation of International Patent Application No. PCT/US2023/017205, filed Mar. 31, 2023, entitled “MEDICAL DELIVERY SHEATH ASSEMBLY,” which claims the benefit of U.S. Provisional Patent Application No. 63/327,034, filed Apr. 4, 2022, entitled “MEDICAL DELIVERY SHEATH ASSEMBLY,” the disclosures of each of which are hereby expressly incorporated by reference herein in their entireties for all purposes.

### BACKGROUND

The present disclosure generally relates to the field of minimally invasive transcatheter delivery of medical devices. Minimally invasive transcatheter medical procedures can comprise delivery of multiple medical devices to different target locations within a patient. Multiple medical devices can be simultaneously disposed within the body of the patient during a procedure. The procedures can be used to treat a variety of conditions, including conditions of the heart.

### SUMMARY

Described herein are methods and devices related to a medical delivery sheath assembly configured to allow disposing multiple medical devices within a patient simultaneously via one common access opening. In some instances, the medical delivery sheath can comprise a hub member and a shaft extending distally from the hub member. The hub member can comprise a first hub portion and a second hub portion each comprising an opening on a proximal end configured to allow insertion therethrough of a first medical device and a second medical device, respectively. At least a portion of the shaft can be inserted through an access opening formed on a vessel and/or lumen of the patient. Corresponding portions of the first and second medical devices can be advanced through the hub member and into the shaft to thereby allow delivery of both medical devices into the patient via the one access opening. In some instances, a medical delivery sheath assembly can comprise a shaft with a bifurcated proximal portion. Distally oriented portions of a first and second proximal shaft portion can be joined to one another and coupled to a common shaft portion. A respective hub member can be coupled to proximally oriented portions of the first and second proximal shaft portions to allow insertion of first and second medical devices into the shaft and delivery of both medical devices into the patient via the one access opening.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 shows an example of a minimally invasive transcatheter delivery of a first medical device and a second medical device to their respective target heart locations via one access opening on the patient.

FIG. 2A is a side view, and FIG. 2B is a longitudinal cross-sectional view, of the medical delivery sheath assembly described with reference to FIG. 1.

FIGS. 3A and 3B are lateral cross-sectional views of the expandable shaft portion of the medical delivery sheath assembly described with reference to FIGS. 1 and 2 in a first expanded state and a second expanded state, respectively.

FIG. 4 is a longitudinal cross-sectional view of an example of a medical delivery sheath assembly that includes a second hub portion comprising a valve with a compressible seal member.

FIG. 5 is a longitudinal cross-sectional view of an example of a medical delivery sheath assembly that includes a second hub portion comprising a one-way valve.

FIG. 6A is a side view, and FIG. 6B is a longitudinal cross-sectional view, of another example of a medical delivery sheath assembly.

FIGS. 7-1 and 7-2 show a process flow diagram illustrating an example of a process for delivering a first and a second medical device to a target location using a medical delivery sheath assembly as described herein.

FIGS. 8-1, 8-2 and 8-3 show images of examples of anatomy and devices associated with the process of FIG. 7 to illustrate aspects of the process.

FIG. 9A is a side view of an example of a medical delivery sheath assembly comprising a shaft with a proximal shaft portion that is bifurcated, and FIG. 9B is a lateral cross-sectional view of a portion of the shaft.

FIGS. 10A and 10B are side views of the medical delivery sheath assembly described with reference to FIG. 9 in a first expanded state and a second expanded state, respectively.

FIG. 11 is a process flow diagram illustrating an example of a process for delivering a first and a second medical device to a target location using a medical delivery sheath assembly as described herein.

### DETAILED DESCRIPTION

The headings provided herein are for convenience only and do not necessarily affect the scope or meaning of the claimed invention.

The present disclosure provides systems, devices, and methods relating to medical delivery sheath assemblies configured to allow disposing multiple medical devices within a patient simultaneously via one common access opening formed on the patient. A medical delivery sheath assembly as described herein can facilitate delivery of a first medical device and a second medical device to respective locations within the patient, including locations in different vessels, lumens, organs, and/or chambers, via one access opening formed on the patient. In some instances, the medical delivery sheath can include a hub member comprising a first hub portion and a second hub portion each with an opening on a proximal end configured to allow insertion therethrough of the first medical device and the second medical device, respectively. A shaft extending distally from the hub member can comprise at least a portion configured to be inserted through the access opening such that corresponding portions of the first and second medical devices can be advanced through the hub member and into the shaft to thereby allow delivery of both medical devices into the patient via the one access opening. In some instances, a medical delivery sheath assembly can comprise a shaft with a bifurcated proximal portion. Distally oriented portions of a first and second proximal shaft portion can be joined together and coupled to a common shaft portion. Proximally oriented portions of the first and second proximal shaft portions can be coupled to a respective hub member to allow insertion of first and second medical devices into the shaft. At least a portion of the common shaft portion can be inserted through the access opening formed on the patient such that both medical devices can be inserted into the patient via the one access opening.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Some minimally invasive transcatheter medical procedures can comprise delivery of multiple medical devices into a patient. Medical procedures for treating a number of conditions can require and/or benefit from being able to position multiple medical devices simultaneously within the body of the patient. The medical devices may each need to be delivered to their respective target locations in different vessels, lumens, organs, and/or chambers of the patient. Minimally invasive transcatheter medical procedures can be used to treat a number of heart conditions. Multiple medical devices may need to be positioned into the patient during the medical procedures, including one or more medical devices for treatment of the heart conditions, and one or more medical devices to support heart function. For example, a medical device to facilitate blood flow through and/or from the heart can be positioned in the heart during at least a portion of the medical procedure while one or more medical devices are delivered to their respective target locations for treatment of the heart condition.

In some medical procedures for improving blood flow through a coronary artery, such as to treat a coronary artery blockage, a mechanical circulatory support (MCS) device can be delivered to the heart to support blood flow. For example, a percutaneous coronary intervention (PCI) procedure can comprise delivery to the heart of both a mechanical circulatory support (MCS) device and a medical device for treatment of the coronary artery blockage. Patients with increased risk for complications, including patients which severe heart disease, can benefit from and/or require use of a mechanical circulatory support (MCS) device to support blood flow while one or more medical devices to improve blood flow through the coronary artery are delivered to their respective target locations. Typical percutaneous coronary intervention (PCI) procedures can require delivery of the mechanical circulatory support (MCS) device through one access opening and the one or more medical devices to improve blood flow through the coronary artery via a second access opening. For example, the mechanical circulatory support (MCS) device can be inserted through an access opening on a left femoral artery and one or more medical devices configured to improve blood flow through the coronary artery can be inserted through an access opening on a right femoral artery, or vice versa.

The disclosure herein provides one or more devices and methods related a medical delivery sheath assembly configured to allow disposing multiple medical devices within a patient simultaneously via one common access opening. For example, the medical delivery sheath assembly can be configured to deliver a first medical device and a second medical device to respective target locations. The target locations can be in the same or different vessels, lumens, organs, and/or chambers. In some instances, the first and second medical devices can be delivered to different target locations in different vessels, lumens, organs, and/or chambers.

In some instances, the medical delivery sheath assembly can comprise a hub member and a shaft extending distally from the hub member. At least a portion of the shaft can be inserted through an access opening formed on a vessel and/or lumen of the patient, and advanced into the vessel and/or lumen. The hub member can comprise a first hub portion and a second hub portion each comprising an opening on a proximal end configured to allow insertion therethrough of a first medical device and a second medical device, respectively. A joint hub portion can be disposed distally of and coupled to respective distal ends of the first and second hub portions. The shaft can extend distally from a distal end of the joint hub portion. The first medical device can be advanced from the first hub portion into the joint hub portion, and then the shaft. The second medical device can be advanced from the second hub portion into the joint hub portion, and then the shaft. The medical delivery sheath assembly can therefore facilitate positioning of two medical devices into the patient via only one access opening. A longitudinal axis of the first hub portion and a longitudinal axis of the second hub portion can form an acute angle where the distal portion of the first hub portion and the distal portion of the second hub portion couple to and/or merge with one another. For example, delivery lumens extending through the respective first and second hub portions can form an acute angle. The acute angle can be selected to facilitate insertion therethrough of the first and second medical devices to reduce or prevent undesired bending and/or kinking of the medical devices.

In some instances, a medical delivery sheath assembly can comprise a shaft with a bifurcated proximal portion. A first and second proximal shaft portion of the shaft can be joined and/or merged together. A common shaft portion can extend distally from where the first and second proximal shaft portions join and/or merge together. For example, distally oriented portions the first and second proximal shaft portions can be joined together and coupled to the common shaft portion. Proximally oriented portions of the first and second proximal shaft portions can be coupled to a respective hub member to allow insertion of first and second medical devices into the shaft. At least a portion of the common shaft portion can be inserted the common access opening such that both medical devices can be inserted into the patient via the one access opening. In some instances, the first proximal shaft portion and the second proximal shaft portion can be flexibly joined together. For example, while in a relaxed state, longitudinal axes of the first and second proximal shaft portions, including longitudinal axes of a delivery lumen extending through the respective proximal shaft portion, can form an acute angle. The first proximal shaft portion and the second proximal shaft portion can be flexibly joined together such that an orientation of the first proximal shaft portion and/or second proximal shaft portion can change while an operator is manipulating the medical delivery sheath assembly. In some instances, the shaft can comprise a shape-memory material, such as a braided shape-memory material. For example, the shaft can comprise a braided nitinol material forming at least the bifurcation of the shaft.

In some instances, a medical delivery sheath assembly as described herein can be used in a percutaneous coronary intervention (PCI) procedure. In some instances, the first medical device can comprise a mechanical circulatory support (MCS) device. For example, the mechanical circulatory support (MCS) device, such as a blood pump, can be used to assist blood flow through and/or from the heart during the percutaneous coronary intervention (PCI) procedure. The second medical device can comprise a medical device configured to address reduced blood flow through the coronary artery. In some instances, the second medical device can comprise a stent. The stent can be positioned within a portion of the coronary artery with reduced blood flow so as to improve and/or restore blood flow therethrough. In some instances, the mechanical circulatory support (MCS) device can be delivered to the heart, including the left heart to assist blood flow into the aorta for delivery to the rest of the body. For example, at least a portion of the mechanical circulatory support (MCS) device can be positioned within the left ventricle to assist delivery of blood to the rest of the body while the stent is delivered to a target location within a coronary artery. Rather than inserting the stent and the mechanical circulatory support (MCS) device into the patient using two different access openings, both the stent and the mechanical circulatory support (MCS) device can be delivered via one access opening, such as an access opening formed on the right femoral artery or the left femoral artery.

It should be understood that medical delivery sheath assemblies described herein can comprise a hub member having more than two hub portions and/or a shaft with a proximal portion having more than two proximal shaft portions. For example, a hub member can comprise three or more hub portions, each hub portion being configured to allow advancement therethrough of a respective medical device. In some instances, a proximal portion of a shaft can be split into three or more proximal shaft portions, each proximal shaft portion being configured to allow advancement therethrough of a respective medical device

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

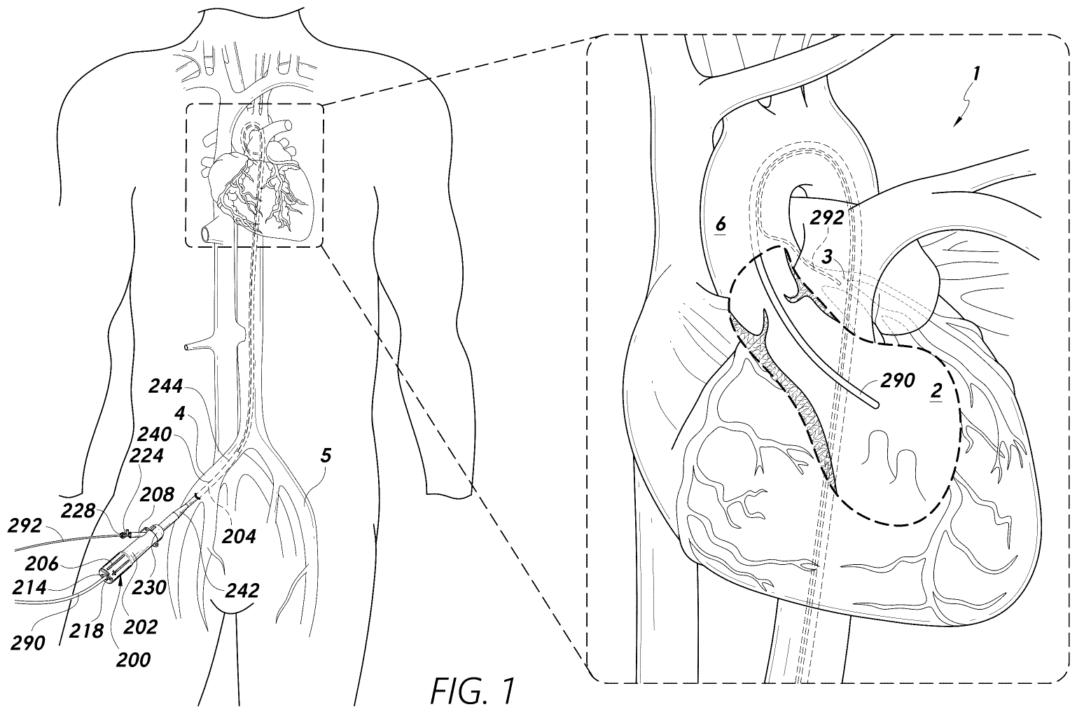

As described herein, certain procedures may require multiple medical devices to be disposed within a patient at the same time. FIG. 1 shows an example of a minimally invasive transcatheter delivery of a first medical device 290 and a second medical device 292 to their respective target locations in and/or on a heart 1 via one access opening on the patient. In some instances, the access opening can be formed on the right femoral artery 4. It will be understood that the location of the access opening is shown for illustrative purposes only. The access opening can be made on any number of vessels and/or lumens of the patient, including for example, the left femoral artery 5. In some instances, the access opening can be made on another arterial blood vessel. In some instances, the access opening can be made on a venous blood vessel, including a femoral vein, jugular vein or subclavian vein. As described in further detail herein, delivering the first and second medical devices 290, 292 to their respective target heart locations can comprise advancement of at least a portion of each of the first and second medical devices 290, 292 through a medical delivery sheath assembly 200. The medical delivery sheath assembly 200 can comprise a hub member 202 and a shaft 204 extending distally from the hub member 202. At least a portion of the shaft 204 can be advanced through the access opening and into the right femoral artery 4. The hub member 202 can comprise a first hub portion 206 and a second hub portion 208. A proximal end 214 of the first hub portion 206 can comprise an insertion opening 218 to allow advancement therethrough of the first medical device 290. A proximal end 224 of the second hub portion 208 can comprise an insertion opening 228 to allow advancement therethrough of the second medical device 292. The hub member 202 can comprise a joint hub portion 230 disposed distally of and coupled to the first and second hub portions 206, 208. The shaft 204 can extend distally from the joint hub portion 230. The joint hub portion 230 and the shaft 204 can be configured to receive corresponding portions of both the first and second medical devices 290, 292. The medical delivery sheath assembly 200 can therefore facilitate positioning of both the first and second medical devices 290, 292 into the patient via one access opening.

In some instances, the shaft 204 can comprise at least a portion that is expandable. For example, the shaft 204 can comprise an expandable shaft portion 240 configured to expand to accommodate both the first and second medical devices 290, 292. The expandable shaft portion 240 can be configured to assume a first expanded state while at least a portion of the first medical device 290 is positioned therethrough. The expandable shaft portion 240 can be configured to assume a second expanded state while at least a portion of both the first medical device 290 and at least a portion of the second medical device 292 are positioned therethrough. For example, the expandable shaft portion 240 can transform from a collapsed state to the first expanded state as corresponding portions of the first medical device 290 is advanced through the expandable shaft portion 240 so as to accommodate the first medical device 290. The expandable shaft portion 240 can transform from the first expanded state to the second expanded state as corresponding portions of the second medical device 292 is advanced through the expandable shaft portion 240 so as to accommodate both the first medical device 290 and the second medical device 292.

A distal portion 244 of the shaft 204 can be advanced to a desired location within the patient. In some instances, the distal portion 244 of the shaft 204 can remain in the right femoral artery 4. In some instances, a dilator (not shown) can be preloaded within the shaft 204 to facilitate navigation of the distal portion 244 of the shaft 204 to the desired location. In some instances, the dilator can be preloaded through the first hub portion 206. For example, corresponding portions of the dilator can be disposed within the first hub portion 206, the joint hub portion 230, and the shaft 204. The dilator can be withdrawn and removed from the medical delivery sheath assembly 200 after the shaft 204 is desirably positioned within the patient. At least a portion of the first medical device 290 can then be advanced into the first hub portion 206 and from the first hub portion 206 into the joint hub portion 230. A corresponding portion of the first medical device 290 can then be advanced from the joint hub portion 230 into and through the shaft 204. In some instances, while the first medical device 290 is advanced to its target location, the second hub portion 208 can be closed and/or sealed. In some instances, at least a portion of the first medical device 290 can be navigated from the right femoral artery 4 into the aorta 6 to facilitate delivery of the first medical device 290 into a heart chamber. For example, at least a portion of the first medical device 290 can be advanced into the left ventricle 2 and positioned at its target site in the left ventricle 2.

In some instances, after the first medical device 290 is at a target position within the patient, at least a portion of the second medical device 292 can be advanced into the second hub portion 208. A corresponding portion of the second medical device 292 can be advanced from the second hub portion 208 into the joint hub portion 230 and through the joint hub portion 230 into the shaft 204. At least a portion of the second medical device 292 can be advanced through the shaft 204 and navigated from the right femoral artery 4 into the aorta 6 to facilitate delivery of the second medical device 292 into a coronary artery 3 to its target site in the coronary artery 3.

In some instances, the first medical device 290 can comprise a Mechanical Circulatory Support (MCS) device, such as a pump, configured to improve blood flow out of the left ventricle 2 and into the aorta 6. In some instances, the second medical device 292 can comprise a medical device, including a stent, configured to be positioned within the coronary artery 3 to improve blood flow therethrough.

Use of the medical delivery sheath assembly 200 can therefore facilitate advancement of both the first medical device 290 and the second medical device 292 to a target site through a common access opening formed on the femoral artery 4. Use of one access opening to introduce multiple medical devices that need to be positioned within the patient simultaneously can simplify the medical procedure, reducing procedural duration and patient health complications.

It will be understood that although description is provided relating to advancement of first and second medical devices to their respective target sites, the first and second medical devices can be advanced to their respective target sites carried on and/or in any number of different delivery catheters and/or cannulas. Description herein can apply to advancement, insertion, and/or positioning of respective delivery catheters through a medical delivery sheath assembly to facilitate simultaneous positioning of multiple medical devices at different target locations within a patient. The order of insertion of the medical devices is for illustrative purposes only. In some instances, a second medical device can be advanced into a medical delivery sheath assembly before a first medical device is advanced into the medical delivery sheath assembly. In some instances, a first medical device can be inserted through a second hub portion and a second medical device can be inserted through a first hub portion.

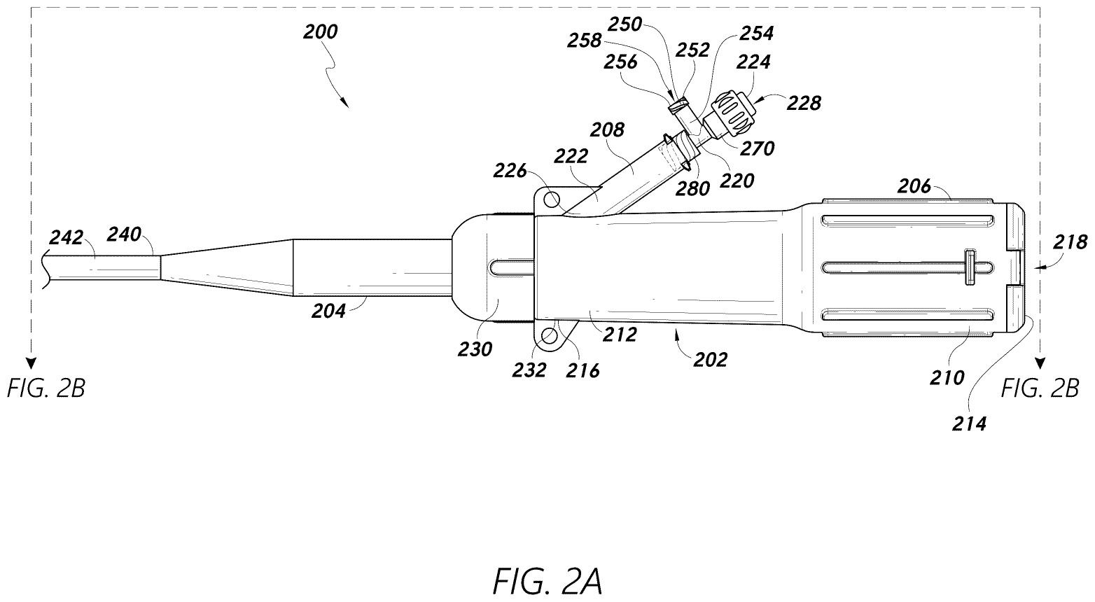

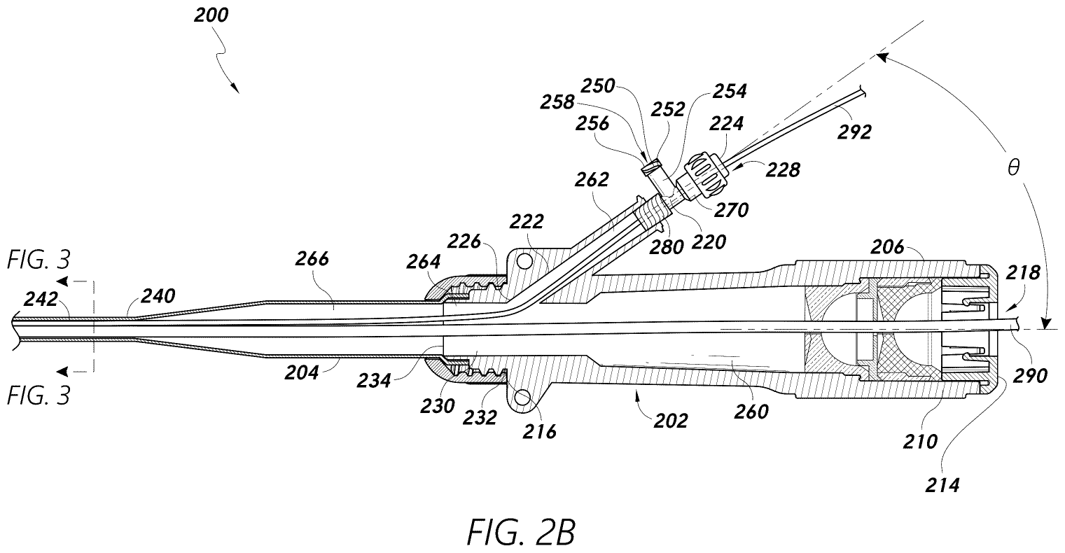

FIGS. 2A and 2B show in further detail the medical delivery sheath assembly 200 described with reference in FIG. 1. FIG. 2A is a side view, and FIG. 2B is a longitudinal cross-sectional view, of the medical delivery sheath assembly 200. Referring to FIG. 2A, the medical delivery sheath assembly 200 can comprise the hub member 202 and the shaft 204. The hub member 202 can comprise the first hub portion 206 and the second hub portion 208. Each of the first hub portion 206 and the second hub portion 208 can comprise a respective proximal portion 210, 220 and a respective distal portion 212, 222. The distal portion 212 of the first hub portion 206 and the distal portion 222 of the second hub portion 208 can be coupled to one another. A joint hub portion 230 can be disposed distally of the first hub portion 206 and the second hub portion 208. A proximal end 232 of the joint hub portion 230 can be coupled to the distal end 216 of the first hub portion 206 and the distal end 226 of the second hub portion 208. For example, the distal portion 212 of the first hub portion 206 can merge with the distal portion 222 of the second hub portion 208 such that proximal end 232 of the joint hub portion 230 can be coupled to and/or be merged with the distal end 216 of the first hub portion 206 and the distal end 226 of the second hub portion 208. In some instances, the first, second and joint hub portions 206, 208, 230 can be integrally formed. For example, the hub member 202 can be an integrally formed hub and comprise the first, second and joint hub portions 206, 208, 230. Alternatively, one or more of the first, second and joint hub portions 206, 208, 230 can be separably coupled to one or more of the other hub portions.

In some instances, the first and second hub portions 206, 208 can each comprise one or more of any number of types of valves configured to prevent backflow of fluid around any medical device inserted therethrough, including hemostatic valves. In some instances, the second hub portion 208 of the medical delivery sheath assembly 200 can comprise an adjustable valve 270 having a compressible seal member. The compressible seal member can comprise a central lumen extending therethrough along a longitudinal axis, the central lumen being configured to receive a corresponding portion of the second medical device. For example, the longitudinal axis of the central lumen can be parallel or substantially parallel and/or coaxial with a longitudinal axis of the second hub portion 208. The central lumen can be configured to reduce in diameter as the compressible seal member is compressed along the longitudinal axis such that portions of the compressible seal member defining the central lumen can contact and/or form a seal around the corresponding portion of the medical device extending therethrough. The adjustable valve 270 can comprise a first member and a second member. The first and second members can comprise a respective portion configured to be oriented toward one another. Each of the first and second members can comprise a surface portion configured to contact corresponding portions of the compressible seal member, such as a proximal or a distal surface portion of the compressible seal member. A dimension of the compressible seal member extending along the longitudinal axis can be reduced as one or both of the first and second member are moved toward the other. For example, a cross-sectional size of the central lumen taken along a plane perpendicular to the longitudinal axis of the compressible seal member can be reduced as the compressible seal member is compressed along the longitudinal axis. The reduction in the cross-sectional size of the central lumen can cause inner lumen wall defining the central lumen to contact and form a seal around exterior surfaces of the portion of the medical device extending through the central lumen. In some instances, the adjustable valve 270 can comprise a Tuohy Borst valve.

The medical delivery sheath assembly 200 can comprise a flush port 250 associated with the second hub portion 208. For example, the flush port 250 can be on and/or extend from the second hub portion 208. The flush port 250 can comprise a proximal portion 252 having an opening 258 on a proximal end 256 in communication with a flush lumen 268 (not shown) extending through the flush port 250 to allow provision therethrough of a flushing fluid. A distal portion 254 of the flush port 250 can be coupled to and/or merge with the second hub portion 208. The flush port 250 can merge with and/or couple to the second hub portion 208 at a variety of different positions on the second hub portion 208. The flush lumen 268 can be in fluid communication with a second delivery lumen 262 extending through the second hub portion 208. For example, the flush lumen 268 can be in direct fluid communication with the second delivery lumen 262, merging with the second delivery lumen 262. The flush port 250 can comprise one or more of any number of different types of seals configured to prevent backflow of fluid around any medical instrument inserted therethrough for delivering the flushing fluid, including hemostatic valves. For example, a distal portion of a syringe can be inserted through the opening 258 on the proximal end 256 and into the flush lumen 268 to deliver flushing fluid into the flush lumen 268.

In some instances, the flush port 250 and the portion of the second hub portion 208 comprising the adjustable valve 270 can be integrate into a separable member. The separable member can be separated from a remaining portion of the second hub portion 208. For example, the portion of the second hub portion 208 comprising the adjustable valve 270 and the portion of the second hub portion 208 from which the flush port 250 extends can be an integrated unit that is separable from the remaining portion of the second hub portion 208, such as a distal portion 222 of the second hub portion 208. For example, the proximal portion 220 of the second hub portion 208 can comprise the adjustable valve 270 and the flush port 250 associated therewith. The proximal portion 220 of the second hub portion 208 can be separable from the distal portion 222 of the second hub portion 208. In some instances, the separable member can comprise a plurality of threads 280 configured to be mated to the remaining portion of the second hub portion 208. Alternatively, the proximal and distal portions 220, 222 of the second hub portion 208 can be integrated and may not be separable from one another.

Referring to FIG. 2B, the first and second medical devices 290, 292 are shown as being slidably received by the medical delivery sheath assembly 200. The first hub portion 206 can comprise a first delivery lumen 260 extending through the first hub portion 206 and configured to slidably receive at least a portion of the first medical device 290. The second hub portion 208 can comprise a second delivery lumen 262 extending therethrough and configured to slidably receive at least a portion of the second medical device 292. As used herein, a “delivery lumen” can refer to any number of lumens, channels, passages, and/or conduits through which a medical instrument and/or medical device can be advanced, including for example a working channel. For example, a “delivery lumen” as used herein can refer to a working channel through which a medical instrument and/or medical device can be advanced for positioning the medical instrument and/or medical device at a target site within a patient. A proximal end 214 of the first hub portion 206 can comprise an insertion opening 218 at a proximal end of the first delivery lumen 260 to allow advancement therethrough of the first medical device 290. A proximal end 224 of the second hub portion 208 can comprise an insertion opening 228 at a proximal end of the second delivery lumen 262 to allow advancement therethrough of the second medical device 292. In some instances, the first medical device 290 can comprise a mechanical circulatory support (MCS) device. The second medical device 292 can comprise a percutaneous coronary intervention (PCI) device. For example, the first delivery lumen 260 can be configured to slidably receive at least a portion of a mechanical circulatory support (MCS) device. The second delivery lumen 262 can be configured to slidably receive at least a portion of a percutaneous coronary intervention (PCI) device, such as a stent.

A longitudinal axis of the first hub portion 206 can be at an angle relative to a longitudinal axis of the second hub portion 208. For example, the longitudinal axis of the first hub portion 206 and the longitudinal axis of the second hub portion 208 can form an acute angle where the distal portion 212 of the first hub portion 206 and the distal portion 222 of the second hub portion 208 couple to and/or merge with one another. This angle is shown as “Θ” in FIG. 2B. In some instances, the acute angle can be between about 10° and about 60°. In some instances, the acute angle can be between about 15° and about 45°. The angle “Θ” can be selected based at least on desired advancement of a respective medical device from the first hub portion 206 and the second hub portion 208 into the joint hub portion 230 and shaft 204. The angle “Θ” can be selected to reduce or prevent undesired bending and/or kinking of the medical devices.

In some instances, a longitudinal axis of the first delivery lumen 260 and a longitudinal axis of the second delivery lumen 262 can form an acute angle where a distal portion 212 of the first hub portion 206 couples to and/or merges with a distal portion 222 of the second hub portion 208. In some instances, the acute angle can be between about 10° and about 60°. In some instances, the acute angle can be between about 15° and about 45°. In some instances, the first delivery lumen 260 can be parallel or substantially parallel and/or coaxial with the longitudinal axis of the first hub portion 206. In some instances, the second delivery lumen 262 can be parallel or substantially parallel and/or coaxial with the longitudinal axis of the second hub portion 208. For example, the angle formed by the longitudinal axes of the first and second delivery lumens 260, 262 can be the same as the angle “Θ”.

The joint hub portion 230 can comprise a third delivery lumen 264 extending therethrough and configured to slidably receive corresponding portions of each the first medical device 290 and the second medical device 292. The third delivery lumen 264 can be in fluid communication with the first and second delivery lumens 260, 262. For example, the first medical device 290 can be advanced through the insertion opening 218 on the first hub portion 206 into the first delivery lumen 260. The first medical device 290 can be advanced through the first delivery lumen 260 and into the third delivery lumen 264. The second medical device 292 can be advanced through the insertion opening 228 on the second hub portion 208 into the second delivery lumen 262. The second medical device 292 can be advanced through the second delivery lumen 262 and into the third delivery lumen 264. Corresponding portions of the first and second medical devices 290, 292 can be received within the third delivery lumen 264.

In some instances, the longitudinal axis of the first hub portion 206 can be coaxial with a longitudinal axis of the joint hub portion 230. For example, the longitudinal axis of the second hub portion 208 can form the acute angle “Θ” with the longitudinal axis of the joint hub portion 230. In some instances, the third delivery lumen 264 can be coaxial with the longitudinal axis of the joint hub portion 230. For example, the longitudinal axis of the second delivery lumen 262 can form the acute angle “Θ” with the longitudinal axis of the third delivery lumen 264.

As described herein, the shaft 204 can be coupled to the hub member 202. The shaft 204 can extend distally from the hub member 202. For example, the shaft 204 can extend distally from the joint hub portion 230. In some instances, a proximal portion 242 of the shaft 204 can be coupled to a distal end 234 of the joint hub portion 230. The shaft 204 can comprise a shaft delivery lumen 266 extending through the shaft 204 and be configured to slidably receive corresponding portions of the first medical device 290 and/or the second medical device 292. For example, the shaft delivery lumen 266 can be in fluid communication with the third delivery lumen 264 such that respective portions of the first and second medical devices 290, 292 can be advanced through the third delivery lumen 264 and from the third delivery lumen 264 into the shaft delivery lumen 266. As described in further detail herein, in some instances, the shaft 204 can comprise an expandable shaft portion 240 configured to expand to accommodate both the first and second medical devices. In some instances, a longitudinal axis of the shaft 204 can be coaxial with that of the joint hub portion 230 and the first hub portion 206. For example, the longitudinal axis of the shaft 204 and that of the second hub portion 208 can form the acute angle “Θ”. In some instances, a longitudinal axis of the shaft delivery lumen 266 can be coaxial with that of the shaft 204 such that the longitudinal axis of the second hub portion 208 and/or the second delivery lumen 262 can form the acute angle “Θ” with the longitudinal axis of the shaft delivery lumen 266.

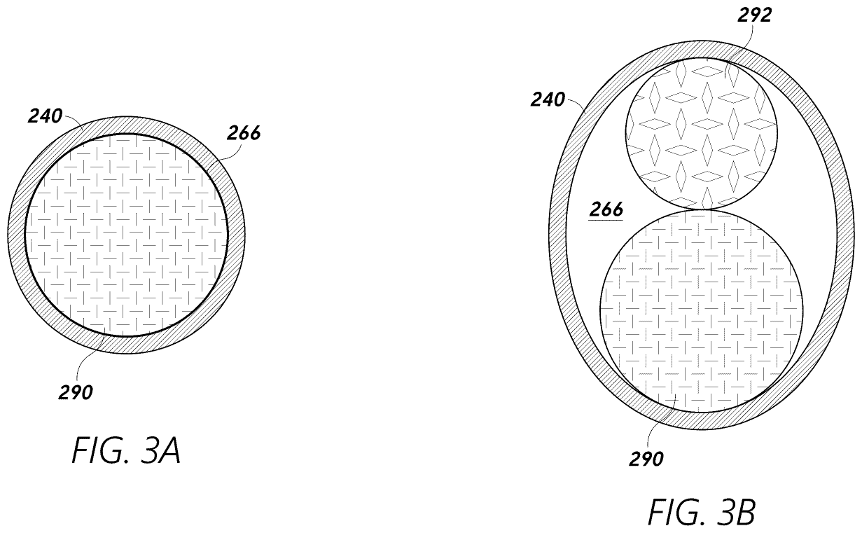

FIGS. 3A and 3B are lateral cross-sectional views of the expandable shaft portion 240 in a first expanded state and a second expanded state, respectively. The lateral cross-section is taken along a plane perpendicular or substantially perpendicular to the longitudinal axis of the shaft 204. Referring to FIG. 3A, the expandable shaft portion 240 can be configured to assume the first expanded state while at least a portion of the first medical device 290 is slidably received within the shaft delivery lumen 266. In some instances, the expandable shaft portion 240 can be configured to expand from a collapsed state to the first expanded state to accommodate the first medical device 290. Referring to FIG. 3B, the expandable shaft portion 240 can be configured to assume the second expanded state while at least a portion of the first medical device 290 and at least a portion of the second medical device 292 are both slidably received within the shaft delivery lumen 266. At least a portion of the first medical device 290 and at least a portion of the second medical device 292 can be configured to be slidably received within a single shaft delivery lumen 266. For example, corresponding portions of the first and second medical devices 290, 292 can be adjacent to and in contact with one another within the shaft delivery lumen 266.

The expandable shaft portion 240 can expand from the first expanded state to the second expanded state to accommodate both the medical devices. In some instances, a lateral cross-section of the expandable shaft portion 240 in the second expanded state can comprise a rounded non-circular shape. The expandable shaft portion 240 can expand to conform to the corresponding portions of the first and/or second medical devices 290, 292 disposed within the shaft delivery lumen 266. For example, the expandable shaft portion 240 in the first expanded state can comprise a lateral cross section configured to conform to a lateral cross-sectional shape and/or size of the first medical device 290. The expandable shaft portion 240 in the second expanded state can comprise a lateral cross section configured to allow the expandable shaft portion 240 to conform to portions of the first and second medical devices 290, 292 configured to contact an inner lumen wall portion of the shaft delivery lumen 266. For example, a lateral cross-sectional shape of the expandable shaft portion 240 in the second expanded state can comprise an oval shape.

An expandable shaft portion can provide a reduced shaft cross-sectional size needed to simultaneously accommodate multiple medical devices that are disposed adjacent to one another, such as compared to a non-expandable shaft. For example, a non-expandable shaft may assume a circular cross-sectional shape. A cross section having a circular shape can have larger area compared to a cross section having an oval shape that has a dimension along a longest axis the same as or similar to the radius of the circular shape. An expandable shaft portion can assume a collapsed state while no medical devices are extended therethrough. A smaller cross-sectional size can improve blood flow in the femoral artery to reduce possible complications during the medical procedures and improve outcome for the patients.

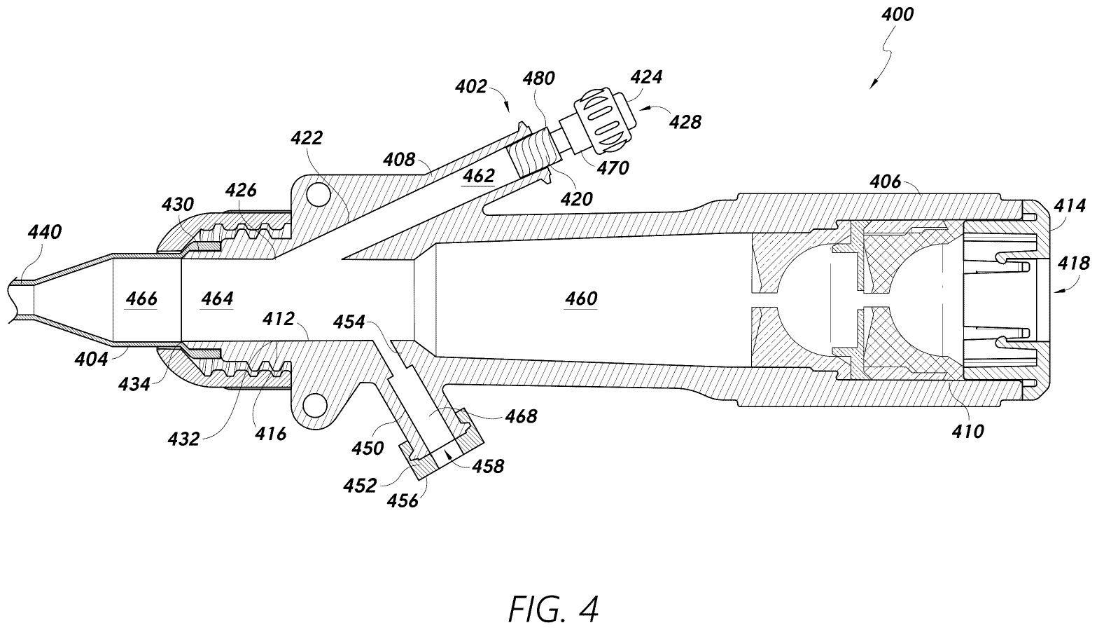

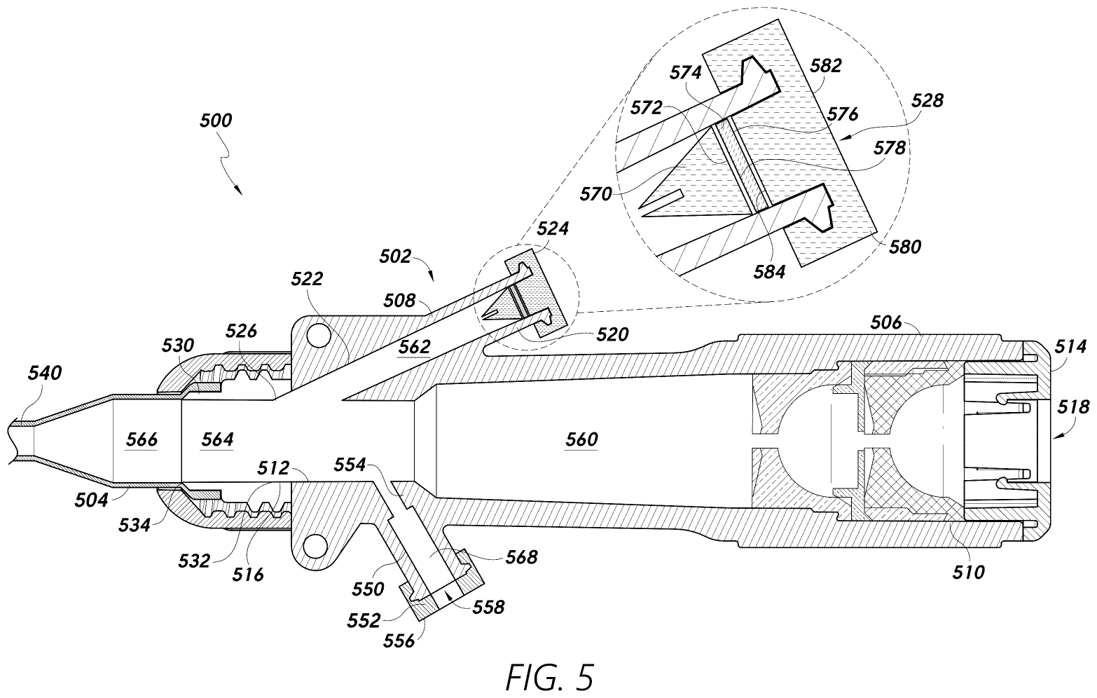

FIGS. 4 and 5 are longitudinal cross-sectional views of examples of medical delivery sheath assemblies 400, 500 that each includes a hub member 402, 502 comprising different types of valves in a second hub portion 408, 508. Each hub member 402, 502 can have a first hub portion 406, 506, the second hub portion 408, 508, and a flush port 450, 550 associated with each of the first hub portions 406, 506. For example, the flush ports 450, 550 can be on and/or extend from the first hub portions 406, 506. Each hub member 402, 502 can comprise a joint hub portion 430, 530 disposed distally of and coupled to respective distal portions 412, 512 of the first hub portion 406, 506 and distal portions 422, 522 the second hub portions 408, 508. A respective shaft 404, 504 can extend distally from the hub members 402, 502. For example, the shafts 404, 504 can extend distally from a distal end 434, 534 of the joint hub portions 430, 530. The first hub portions 406, 506 can comprise a first delivery lumen 460, 560 extending therethrough and configured to slidably receive at least a portion of a first medical device. The second hub portions 408, 508 can comprise a second delivery lumen 462, 562 extending therethrough and configured to slidably receive at least a portion of a second medical device. The joint hub portions 430, 530 can comprise a respective third delivery lumen 464, 564 extending therethrough. The third delivery lumens 464, 564 can be in fluid communication with the first delivery lumens 460, 560 and second delivery lumens 462, 562 and be configured to receive respective portions of both the first and second medical devices. A shaft delivery lumen 466, 566 can extend through the shaft 404, 504 and be in fluid communication with the third delivery lumen 464, 564 to receive respective portions of both the first and second medical devices.

Each of the second hub portions 408, 508 can comprise one or more of any number of different types of valves configured to prevent backflow of fluid around any medical device inserted therethrough, including hemostatic valves. Referring to FIG. 4, the second hub portion 408 of the medical delivery sheath assembly 400 can comprise an adjustable valve 470 having a compressible seal member. The compressible seal member can comprise a central lumen extending therethrough along a longitudinal axis, the central lumen being configured to receive a corresponding portion of a medical device. For example, the longitudinal axis of the central lumen can be parallel or substantially parallel and/or coaxial with a longitudinal axis of the second hub portion 408. The central lumen can be configured to reduce in diameter as the compressible seal member is compressed along the longitudinal axis such that portions of the compressible seal member defining the central lumen can contact and/or form a seal around the corresponding portion of the medical device extending therethrough. The adjustable valve 470 can comprise a first member and a second member. The first and second members can each comprise a respective portion configured to be oriented toward one another. For example, the first and second members can each comprise a surface portion configured to contact corresponding portions of the compressible seal member, such as a proximal or a distal surface portion of the compressible seal member. A dimension of the compressible seal member extending along the longitudinal axis can be reduced as one or both of the first and second member are moved toward the other. For example, a cross-sectional size of the central lumen taken along a plane perpendicular to the longitudinal axis of the compressible seal member can be reduced as the compressible seal member is compressed along the longitudinal axis. The reduction in the cross-sectional size of the central lumen can cause inner lumen wall defining the central lumen to contact and form a seal around exterior surfaces of the portion of the medical device extending through the central lumen. In some instances, the adjustable valve 470 can comprise a Tuohy Borst valve.

In some instances, the portion of the second hub portion 408 comprising the adjustable valve 470 can be integrate into a separable member. The separable member can be separated from a remaining portion of the second hub portion 408. For example, the portion of the second hub portion 408 comprising the adjustable valve 470 can be separable from the remaining portion of the second hub portion 408. For example, a proximal portion 420 of the second hub portion 408 comprising the adjustable valve 470 can be separable from the distal portion 422 of the second hub portion 408. In some instances, the separable member can comprise a plurality of threads 480 configured to be mated to the remaining portion of the second hub portion 408. Alternatively, the proximal and distal portions 420, 422 of the second hub portion 408 can be integrated and may not be separable from one another.

Referring to FIG. 5, the second hub portion 508 of the medical delivery sheath assembly 500 can comprise a one-way valve 570 and/or a disc valve 574. In some instances, one-way valve 570 can comprise a duckbill valve. For example, the second hub portion 508 can comprise both a one-way valve 570 and the disc valve 574. The disc valve 574 can be proximal of the one-way valve 570. For example, a proximal end 572 of the one-way valve 570 can be distal of a distal end 578 of the disc valve 574. In some instances, the disc valve 574 can be proximal of and in contact with the one-way valve 570. instancesFor example, the proximal end 572 of the one-way valve 570 can be adjacent to and in contact with the distal end 578 of the disc valve 574. Corresponding portions of a medical device can be advanced first through the disc valve 574 and then through the one-way valve 574. For example, corresponding portions of a medical device can be advanced first through a central lumen extending through the disc valve 574 and then through a central lumen extending through the one-way valve 570.

In some instances, the second hub portion 508 can comprise a scaling cap 580. In some instances, the sealing cap 580 can form a proximal end 524 of the second hub portion 508. In some instances, the sealing cap 580 can be positioned over a proximal end 576 of the disc valve 574. For example, a distal end 584 of the sealing cap 580 can be oriented toward and/or in contact with the proximal end 576 of the disc valve 574. In some instances, the sealing cap 580 can comprise an insertion opening 528 extending from the proximal end 582 of the scaling cap 580 and through the sealing cap 580. The insertion opening 528 can be configured to allow extension therethrough of a corresponding portion of a medical device. For example, at least a portion of the sealing cap 580 can be positioned over a proximal end of the second delivery lumen 562 such that the sealing cap 580 occludes or substantially occludes the proximal end of the second delivery lumen 562. Alternatively, the sealing cap 580 may not have an opening extending therethrough to receive the medical device. The sealing cap 580 can be configured to be positioned over the disc valve 574 while no medical device is inserted through the second hub portion 508. In some instances, the sealing cap 580 can be configured to seal the proximal end of the second delivery lumen 562 while no medical device is inserted into second delivery lumen 562. The sealing cap 580 can be removed to allow insertion of the medical device.

Referring to FIGS. 4 and 5 again, each of the flush ports 450, 550 can comprise a proximal portion 452, 552 having an opening 458, 558 on a proximal end 456, 556 in communication with a flushing lumen 468, 568 to allow provision therethrough of a flushing fluid. Respective distal portions 454, 554 of the flush ports 450, 550 can be coupled to and/or merge with the respective first hub portion 406, 506. The flush ports 450, 550 can merge with and/or couple to the first hub portions 406, 506 at a variety of different positions on the first hub portions 406, 506. In some instances, the flush ports 450, 550 can merge with and/or couple to a distal portion 412, 512 of the respective first hub portion 406, 506. For example, the flush ports 450, 550 can extend from a respective first hub portion 406, 506, such as the distal portion 412, 512 of the respective first hub portion 406, 506. Each flush lumen 468, 568 can be in fluid communication with the corresponding first delivery lumen 460, 560. For example, each flush lumen 468, 568 can be in direct fluid communication with the corresponding first delivery lumen 460, 560, merging with the corresponding first delivery lumen 460 560. A fluid used for flushing one or more of the lumens of the medical delivery sheath assemblies 400, 500 can flow through the flush lumens 468, 568 into the corresponding first delivery lumen 460, 560. Each of the flush ports 450, 550 can comprise one or more of any number of different types of seals configured to prevent backflow of fluid around any medical instrument inserted therethrough for delivering the flushing fluid, including hemostatic valves. In some instances, a syringe can be used to inject flushing fluid into the flush ports 450, 550. A distal portion of the syringe can be inserted through the opening 458, 558 on the proximal end 456, 556 and into the flush lumen 468, 568 to deliver flushing fluid into the flush lumen 468, 568.

Each of the medical delivery sheath assemblies 400, 500 can have one or more other features of the medical delivery sheath assembly 200 as described with reference to FIGS. 1 through 3. For example, each of the first hub portions 406, 506 can comprise a respective proximal portion 410, 510 and a respective distal portion 412, 512. The second hub portions 408, 508 can comprise a respective proximal portion 420, 520 and a respective distal portion 422, 522. The distal portion 412, 512 of the first hub portions 406, 506 and the distal portion 422, 522 of the second hub portion 408, 508 can be coupled to and/or merge with one another. A longitudinal axis of the first hub portion 406, 506 and a longitudinal axis of the second hub portion 408, 508 can be form an acute angle where the distal portion 412, 512 of the first hub portion 406, 506 and the distal portion 422, 522 of the second hub portion 408, 508 couple to and/or merge with one another. In some instances, the acute angle can be between about 10° and about 60°, including between about 15° and about 45°. In some instances, a longitudinal axis of the first delivery lumen 460, 560 and a longitudinal axis of the second delivery lumen 462, 562 can form an acute angle where a distal portion 412, 512 of the first hub portion 406, 506 couples to and/or merges with a distal portion 422, 522 of the second hub portion 408, 508. In some instances, the longitudinal axis of the first delivery lumen 460, 560 can be coaxial with that of the first hub portion 406, 506. In some instances, the longitudinal axis of the second delivery lumen 462, 562 can be coaxial with that of the second hub portion 408, 508. For example, the acute angle can be between about 10° and about 60°, including about 15° and about 45°.

A proximal end 414, 514 of the first hub portion 406, 506 can comprise an insertion opening 418, 518 at a proximal end of the first delivery lumen 460, 560 to allow advancement therethrough of at least a portion the first medical device. A proximal end 424, 524 of the second hub portion 408, 508 can comprise an insertion opening 428, 528 at a proximal end of the second delivery lumen 462, 562 to allow advancement therethrough of at least a portion of the second medical device. A distal end 416, 516 of the first hub portion 406, 506 and a distal end 426, 526 of the second hub portion 408, 508 can be coupled to and/or merge with a proximal end 432, 532 of the joint hub portion 430, 530. The joint hub portion 430, 530 can comprise a third delivery lumen 464, 564 extending therethrough in fluid communication with the first and second delivery lumens 460, 560, 462, 562 and configured to slidably receive corresponding portions of each the first medical device and the second medical device. The shaft 404, 504 can extend distally from the joint hub portion 430, 530, such as a distal end 434, 534 of the joint hub portion 430, 530. The shaft 404, 504 can comprise an expandable shaft portion 440, 540 being configured to assume a first expanded state while at least a portion of the first medical device is received therein and a second expanded state while at least a portion of both the first medical device and second medical device are received therein.

In some instances, the longitudinal axis of the first hub portion 406, 506 can be coaxial with that of the joint hub portion 430, 530 and shaft 404, 504. For example, the longitudinal axis of the second hub portion 408, 508 and that of the joint hub portion 430, 530 and shaft 404, 504 can be form an acute angle where the longitudinal axes intersect. In some instances, the longitudinal axis of the third delivery lumen 464, 564 and shaft delivery lumen 466, 566 can be coaxial with that of the first delivery lumen 460, 560. The longitudinal axis of the second delivery lumen 462, 562 and that of the third delivery lumen 464, 564 and shaft delivery lumen 466, 566 can form an acute angle where the axes intersect.

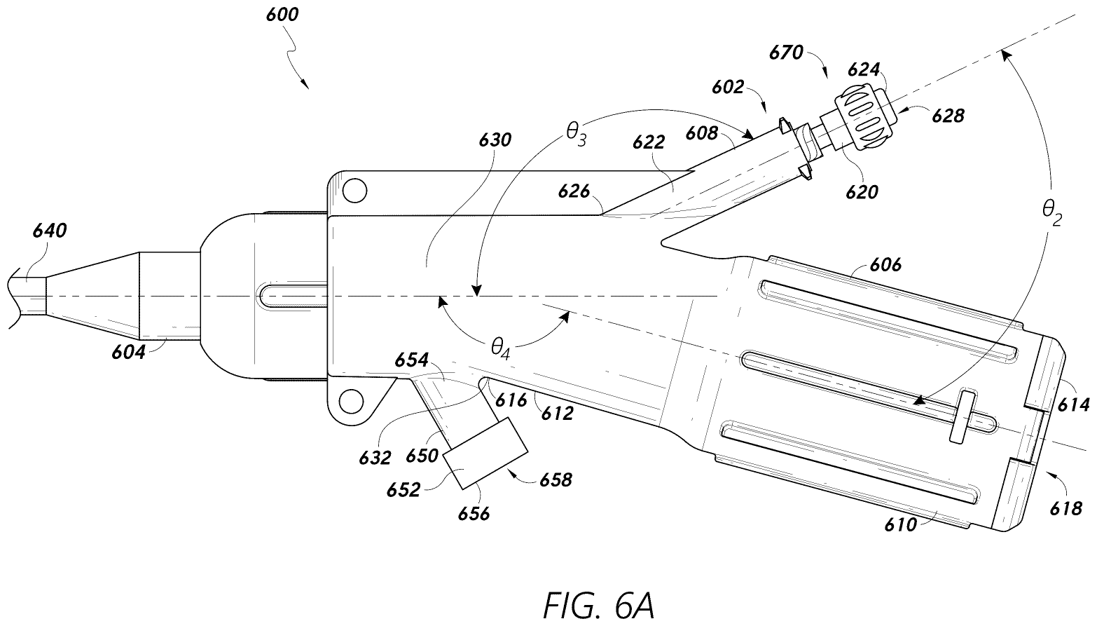

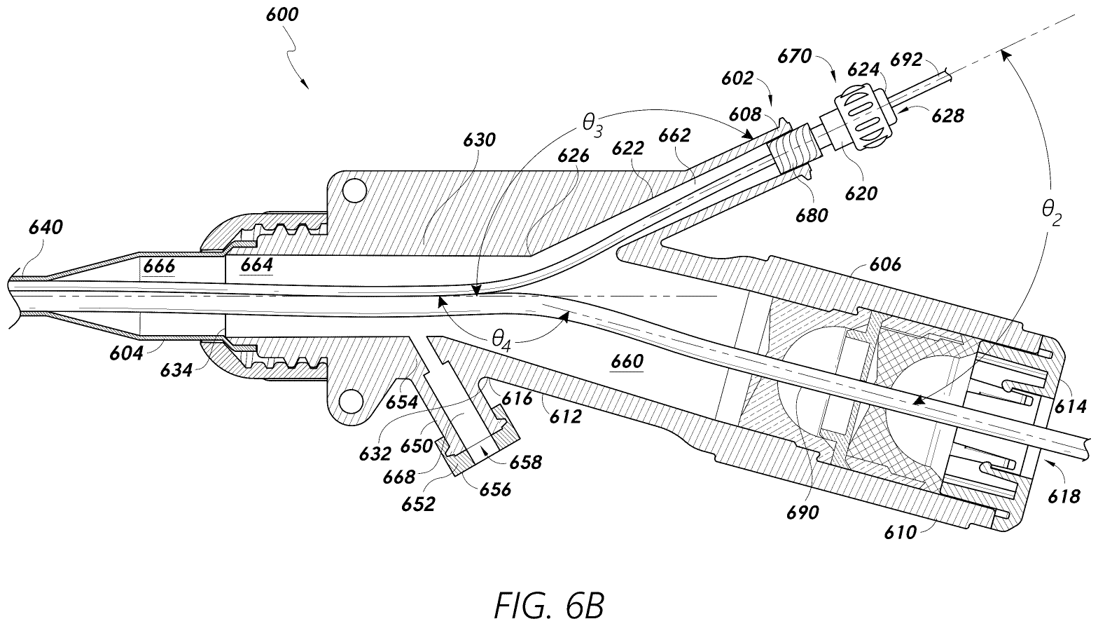

FIGS. 6A and 6B are a side view and a longitudinal cross-sectional view, respectively, of another example of a medical delivery sheath assembly 600. The medical delivery sheath assembly 600 can comprise a hub member 602 and a shaft 604. The hub member 602 can comprise a first hub portion 606 and a second hub portion 608. A joint hub portion 630 can be disposed distally of and coupled to the first hub portion 606 and the second hub portion 608. The shaft 604 can extend distally from the hub member 602. For example, the shaft 604 can extend distally from a distal end 634 of the joint hub portion 630. Both a longitudinal axis of the first hub portion 606 and a longitudinal axis of the second hub portion 608 can be at an angle relative to a longitudinal axis of the joint hub portion 630. A longitudinal axis of the shaft 604 can be parallel or substantially parallel and/or coaxial with that of the joint hub portion 630 such that both the longitudinal axis of the first hub portion 606 and the longitudinal axis of the second hub portion 608 can be at an angle relative to the longitudinal axis of the shaft 604. The longitudinal axis of the first hub portion 606 and that of the second hub portion 608 can form an acute angle where a distal end 616 of the first hub portion 606 and a distal end 626 of the second hub portion 608 couple to and/or merge with one another. The angle is labeled as “Θ₂” in FIGS. 6A and 6B. In some instances, the acute angle can be between about 10° and about 60°. In some instances, the acute angle can be between about 15° and about 45°.

In some instances, an angle formed by the longitudinal axis of the first hub portion 606 and that of the joint hub portion 630, angle labeled as “Θ₄” in FIGS. 6A and 6B, can be the same or similar to an angle formed by the longitudinal axis of the second hub portion 608 and that of the joint hub portion 630, angle labeled as “Θ₃” in FIGS. 6A and 6B. The angle formed by the longitudinal axis of the first hub portion 606 and that of the joint hub portion 630 referred to herein can extend from the longitudinal axis of the first hub portion 606 to that of the joint hub portion 630 without crossing the second hub portion 608. The angle formed by the longitudinal axis of the second hub portion 608 and that of the joint hub portion 630 referred to herein can extend from the longitudinal axis of the second hub portion 608 to that of the joint hub portion 630 without crossing the first hub portion 606.

The first hub portion 606 can comprise a first delivery lumen 660, extending therethrough and configured to slidably receive at least a portion of a first medical device 690. The second hub portion 608 can comprise a second delivery lumen 662 extending therethrough and configured to slidably receive at least a portion of a second medical device 692. The joint hub portion 630 can comprise a respective third delivery lumen 664 extending therethrough and in fluid communication with the first delivery lumen 660 and second delivery lumen 662. The third delivery lumen 664 can be configured to receive respective portions of both the first and second medical devices. A shaft delivery lumen 666 can extend through the shaft 604 and be in fluid communication with the third delivery lumen 664 to receive respective portions of both the first and second medical devices.

A longitudinal axis of the first delivery lumen 660 and a longitudinal axis of the second delivery lumen 662 can form an acute angle where a distal portion 612 of the first hub portion 606 couples to and/or merges with a distal portion 622 of the second hub portion 608. In some instances, the longitudinal axis of the first delivery lumen 660 can be parallel or substantially parallel and/or coaxial with that of the first hub portion 606. In some instances, the longitudinal axis of the second delivery lumen 662 can be parallel or substantially parallel and/or coaxial with that of the second hub portion 608. For example, the acute angle can be between about 10° and about 60°, including about 15° and about 45°. In some instances, an angle formed by the longitudinal axis of the first delivery lumen 660 and that of the third delivery lumen 664 can be the same or similar to an angle formed by the longitudinal axis of the second delivery lumen 662 and that of the third delivery lumen 664. In some instances, the longitudinal axis of the third delivery lumen 664 can be parallel or substantially parallel and/or coaxial with that of the joint hub portion 630. In some instances, the longitudinal axis of the shaft delivery lumen 666 can be parallel or substantially parallel and/or coaxial with that of the shaft 604.

The medical delivery sheath assembly 600 can comprise a flush port 650 associated the joint hub portion 630. For example, the flush port 650 can be on and/or extend from the joint hub portion 630. The flush port 650 can comprise a proximal portion 652 having an opening 658 on a proximal end 656 in communication with a flushing lumen 668 extending through the flush port 650 to allow provision therethrough of a flushing fluid. A distal portion 654 of the flush port 650 can be coupled to and/or merge with the joint hub portion 630. The flush port 650 can merge with and/or couple to the joint hub portion 630 at a variety of different positions on the joint hub portion 630. The flush lumen 668 can be in fluid communication with the first delivery lumen 660. For example, the flush lumen 668 can be in direct fluid communication with the third delivery lumen 664, merging with the corresponding third delivery lumen 664. The flush port 650 can comprise one or more of any number of different types of seals configured to prevent backflow of fluid around any medical instrument inserted therethrough for delivering the flushing fluid, including hemostatic valves. For example, a distal portion of a syringe can be inserted through the opening 658 on the proximal end 656 and into the flush lumen 668 to deliver flushing fluid into the flush lumen 668.

In some instances, the second hub portion 608 can comprise an adjustable valve 670 having a compressible seal member comprising a central lumen extending therethrough along a longitudinal axis. For example, the longitudinal axis of the central lumen can be parallel or substantially parallel and/or coaxial with a longitudinal axis of the second hub portion 608. The central lumen can be configured to receive and form a seal around a corresponding portion of a medical device extending therethrough as the compressible seal is compressed along its longitudinal axis and a diameter of the central lumen is reduced. The adjustable valve 670 can comprise a first member and a second member. The first and second members can each comprise a respective portion configured to be oriented toward one another. For example, the first and second members can each comprise a surface portion configured to contact corresponding portions of the compressible seal member, such as a proximal or a distal surface portion of the compressible seal member. A cross-sectional size of the central lumen taken along a plane perpendicular to the longitudinal axis of the compressible seal member can be reduced as the compressible seal member is compressed along the longitudinal axis. The reduction in the cross-sectional size of the central lumen can cause inner lumen wall defining the central lumen to contact and form a seal around exterior surfaces of the portion of the medical device extending through the central lumen. In some instances, the adjustable valve 670 can comprise a Tuohy Borst valve.

In some instances, the portion of the second hub portion 608 comprising the adjustable valve 670 can be integrate into a separable member. The separable member can be separated from a remaining portion of the second hub portion 608. For example, the portion of the second hub portion 608 comprising the adjustable valve 670 can be separable from the remaining portion of the second hub portion 608. For example, a proximal portion 620 of the second hub portion 608 comprising the adjustable valve 670 can be separable from the distal portion 622 of the second hub portion 608. In some instances, the separable member can comprise a plurality of threads 680 configured to be mated to the remaining portion of the second hub portion 608. Alternatively, the proximal and distal portions 620, 622 of the second hub portion 608 can be integrated and may not be separable from one another.

The medical delivery sheath assembly 600 can have one or more other features of the medical delivery sheath assembly 200 as described with reference to FIGS. 1 through 3. For example, the first hub portion 606 can comprise a respective proximal portion 610 and a distal portion 612. The second hub portion 608 can comprise a proximal portion 620 and a distal portion 622. The distal portion 612 of the first hub portions 606 and the distal portion 622 of the second hub portion 608 can be coupled to and/or merge with one another. A proximal end 614 of the first hub portion 606 can comprise an insertion opening 618 at a proximal end of the first delivery lumen 660 to allow advancement therethrough of at least a portion the first medical device 690. A proximal end 624 of the second hub portion 608 can comprise an insertion opening 628 at a proximal end of the second delivery lumen 662 to allow advancement therethrough of at least a portion of the second medical device 692. A distal end 616 of the first hub portion 606 and a distal end 626 of the second hub portion 608 can be coupled to and/or merge with a proximal end 632 of the joint hub portion 630. The shaft 604 can extend distally from the joint hub portion 630, such as a distal end 634 of the joint hub portion 630. The shaft 604 can comprise an expandable shaft portion 640 being configured to assume a first expanded state while at least a portion of the first medical device 690 is received therein and a second expanded state while at least a portion of both the first medical device 690 and second medical device 692 are received therein.

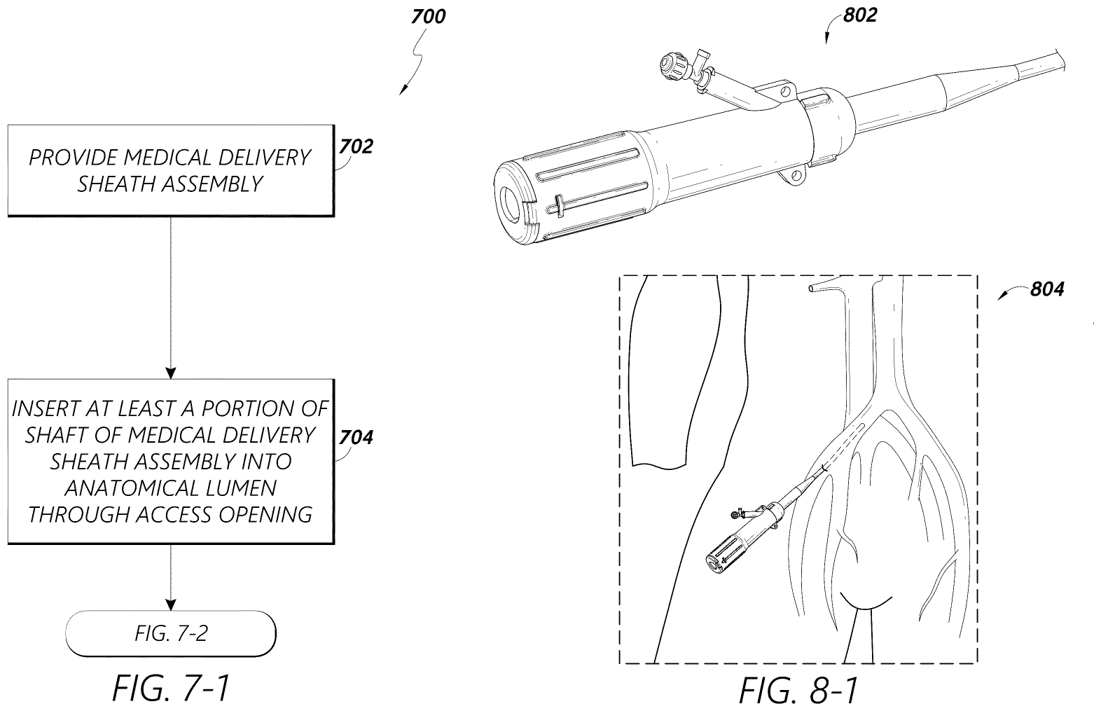

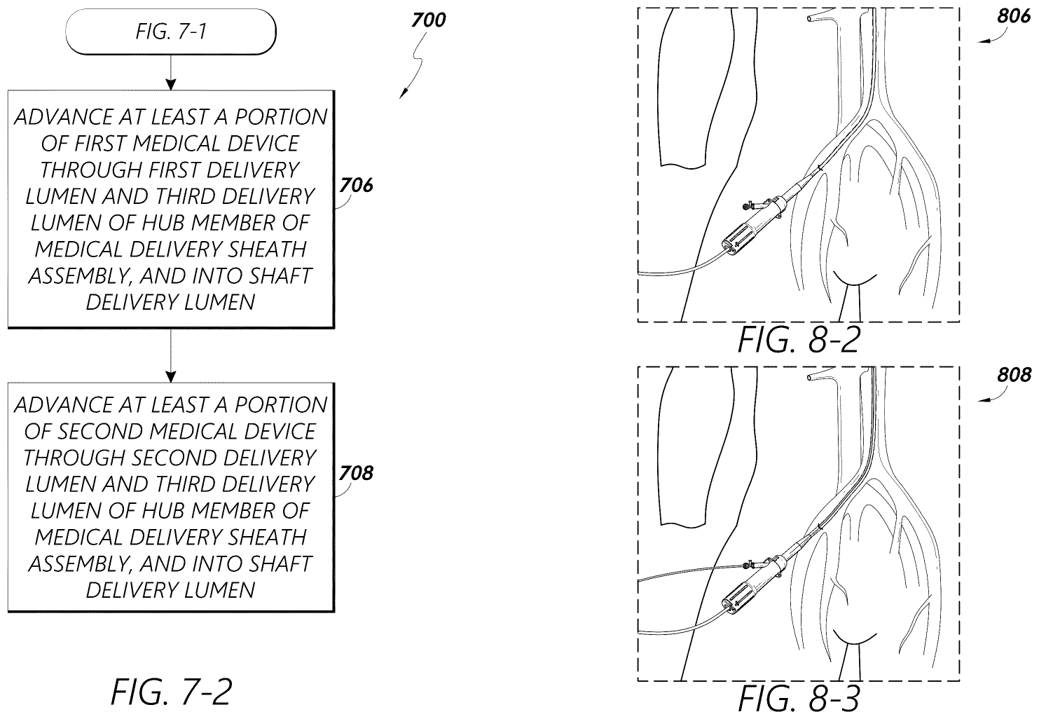

FIGS. 7-1 and 7-2 show a process flow diagram illustrating an example of a delivery process 700 for minimally invasive transcatheter delivery of a first medical device and a second medical device to respective target locations. FIGS. 8-1, 8-2, and 8-3 show images of examples of anatomy and devices associated with respective portions of with the process described with reference to FIG. 7. In block 702, the delivery process can involve providing a medical delivery sheath assembly. For example, the medical device as shown in block 802 of FIG. 8-1 can be provided. The medical delivery sheath assembly can comprise one or more features described herein. For example, the medical delivery sheath assembly can comprise a hub member comprising a first hub portion and a second hub portion. A joint hub portion can be distally disposed from and coupled to a distal portion of the first hub portion and a distal portion of the second hub portion. A shaft can be disposed distally of the joint hub portion, for example extending distally from a distal end of the joint hub portion. The first hub portion can comprise a first delivery lumen. The second hub portion can comprise a second delivery lumen. A longitudinal axis of the first delivery lumen and a longitudinal axis of the second delivery lumen can form an acute angle where a distal portion of the first hub portion merges with a distal portion of the second hub portion. The joint hub portion can comprise a third delivery lumen. The shaft can comprise a shaft delivery lumen. The first delivery lumen and the second delivery lumen can each be in fluid communication with the third delivery lumen. The third delivery lumen can be in fluid communication with the shaft delivery lumen. For example, the first delivery lumen and the second delivery lumen can each be in fluid communication with the shaft delivery lumen via the third delivery lumen.

In block 704, the delivery process can involve inserting at least a portion of the shaft into an anatomical lumen through an access opening. The access opening can be formed on any number of anatomical lumens and/or vessels. In some instances, the anatomical lumen can be a blood vessel, such as a femoral artery. For example, inserting at least a portion of the shaft into the anatomical lumen through the access opening comprises inserting at least a portion of the shaft into an access opening formed on a femoral artery, including a left or a right femoral artery. FIG. 8-1 in block 804 shows insertion of at least a portion of the shaft of the medical delivery sheath assembly in a right femoral artery. In some instances, a venous access site can be used, including a femoral, jugular or sub-clavian venous access site.

In block 706, the delivery process can involve advancing at least a portion of the first medical device through the first delivery lumen and the third delivery lumen, and into the shaft delivery lumen. This is shown in block 806 of FIG. 8-2. In block 708, the delivery process can involve advancing at least a portion of the second medical device through the second delivery lumen and the third delivery lumen, and into the shaft delivery lumen. This is shown in block 808 of FIG. 8-3. As described herein, advancing the first medical device and/or the second medical device into the first delivery lumen, second delivery lumen, respectively, can comprise advancing the first medical device and/or the second medical device carried in and/or on a medical instrument to facilitate their delivery. For example, the first and/or second medical devices can be carried in and/or on a respective delivery catheter. The first and/or second medical devices can be carried in and/or on a respective delivery catheter as they are advanced through the first and second delivery lumen, respectively, and through the third delivery lumen and into the shaft delivery lumen.

The shaft of the medical delivery sheath assembly can comprise at least a portion of which that is expandable. The shaft can comprise an expandable shaft portion configured to assume a collapsed state while being advanced to a target location. For example, the expandable shaft portion can be in the collapsed state before advancement therethrough of the first and second medical devices. In some instances, advancing at least a portion of the first medical device into the shaft delivery lumen can comprise advancing the at least a portion of the medical device into a corresponding portion of the shaft delivery lumen extending through an expandable shaft portion and expanding the expandable shaft portion from a collapsed state to a first expanded state. As the first medical device is advanced into the portion of the shaft delivery lumen extending through the expandable shaft portion, the expandable shaft portion can expand to allow passage therethrough of the first medical device. In some instances, the expandable shaft portion can expand such that an inner diameter of the shaft delivery lumen conforms to an outer diameter of the first medical device. The expandable shaft portion can be in the first expanded state while at least a portion of the first medical device is received therein.

In some instances, advancing at least a portion of the second medical device into the shaft delivery lumen can comprise advancing the at least a portion of the second medical device into a corresponding portion of the shaft delivery lumen extending through the expandable shaft portion and expanding the expandable shaft portion from the first expanded state to a second expanded state. The second expanded state can comprise a lateral cross-sectional size larger than that of the first expanded state. The expandable shaft portion can be in the second expanded state while at least a portion of the first medical device and at least a portion of the second medical device are received therein. Advancing at least a portion of the second medical device into the shaft delivery lumen can comprise advancing the at least a portion of the second medical device into the shaft delivery lumen alongside and adjacent to the at least a portion of the first medical device slidably received in the shaft delivery lumen. In some instances, the expandable shaft portion can expand to the second expanded state to accommodate both the first medical device and the second medical device.

The first and second medical devices can be used in a medical procedure for treating various conditions, including various conditions of the heart. In some instances, the delivery process comprises a minimally invasive percutaneous medical procedure, including a percutaneous coronary intervention (PCI). The percutaneous coronary intervention can comprise transcatheter delivery of both a mechanical circulatory support (MCS) device and a medical device for improvement of blood flow in a coronary artery to their respective target sites. For example, advancing at least a portion of the first medical device through the first delivery lumen and the third delivery lumen, and into the shaft delivery lumen can comprise advancing at least a portion of a mechanical circulatory support (MCS) device through the first delivery lumen and the third delivery lumen, and into the shaft delivery lumen. Advancing at least a portion of a second medical device through the second delivery lumen and the third delivery lumen, and into the shaft delivery lumen can comprise advancing a percutaneous coronary intervention (PCI) device through the first delivery lumen and the third delivery lumen, and into the shaft delivery lumen. At least a portion of the percutaneous coronary intervention (PCI) device can be advanced into a coronary artery. In some instances, the percutaneous coronary intervention (PCI) device can comprise a stent. The stent can be advanced to a target location within a coronary artery to facilitate improvement of blood flow therethrough.

In some instances, the mechanical circulatory support (MCS) device can be configured for left heart support. For example, at least a portion of the mechanical circulatory support (MCS) device can be advanced into a left heart. The mechanical circulatory support (MCS) device can move blood from a left ventricle into an aorta to facilitate supply of oxygenated blood to the rest of the body. For example, at least a portion of the mechanical circulatory support (MCS) device can be advanced into the left ventricle such that the mechanical circulatory support (MCS) device can pump blood from the left ventricle into the aorta. The mechanical circulatory support (MCS) device can be operated during the percutaneous coronary intervention (PCI) procedure to assist blood flow through the heart.

In some instances, advancing at least a portion of the first medical device through the first delivery lumen can comprise advancing the at least a portion of the first medical device through a first delivery lumen comprising a longitudinal axis coaxial to a longitudinal axis of the shaft delivery lumen. In some instances, advancing at least a portion of the second medical device through the second delivery lumen can comprise advancing the at least a portion of the second medical device through a second delivery lumen comprising a longitudinal axis at an angle relative to the longitudinal axis of the shaft delivery lumen.

In some instances, advancing at least a portion of the first medical device through the first delivery lumen can comprise advancing the at least a portion of the first medical device through a first delivery lumen comprising a longitudinal axis at a first angle relative to a longitudinal axis of the shaft delivery lumen. In some instances, advancing at least a portion of the second medical device through the second delivery lumen can comprise advancing the at least a portion of the second medical device through a second delivery lumen comprising a longitudinal axis at a second angle relative to the longitudinal axis of the shaft delivery lumen.

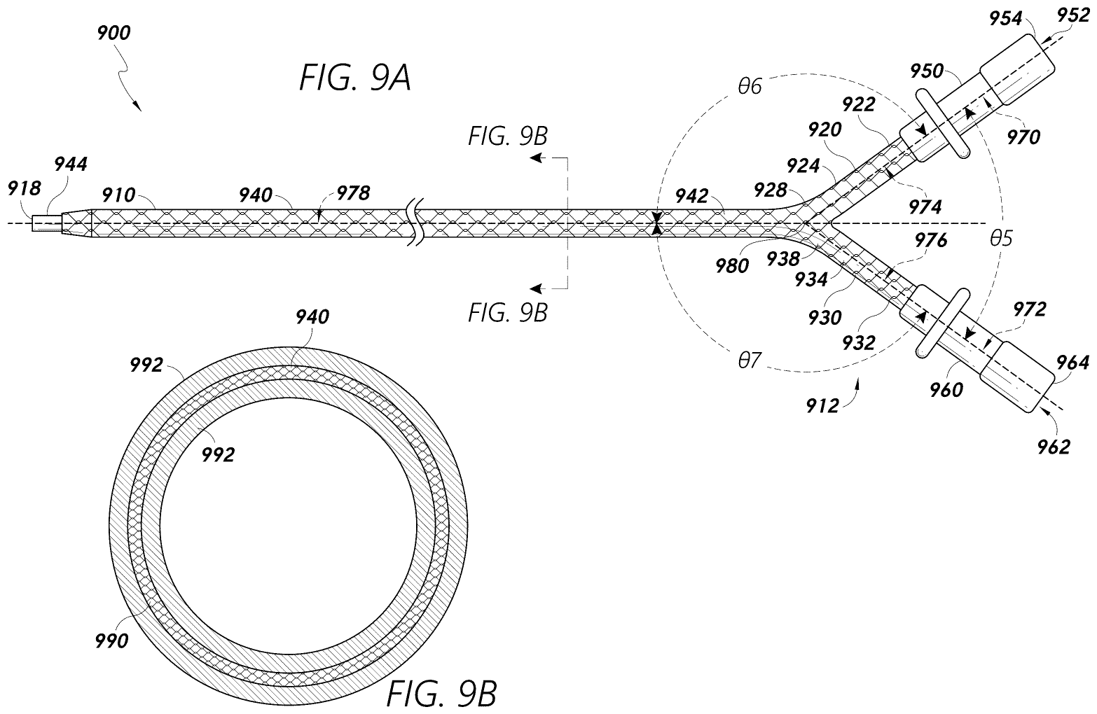

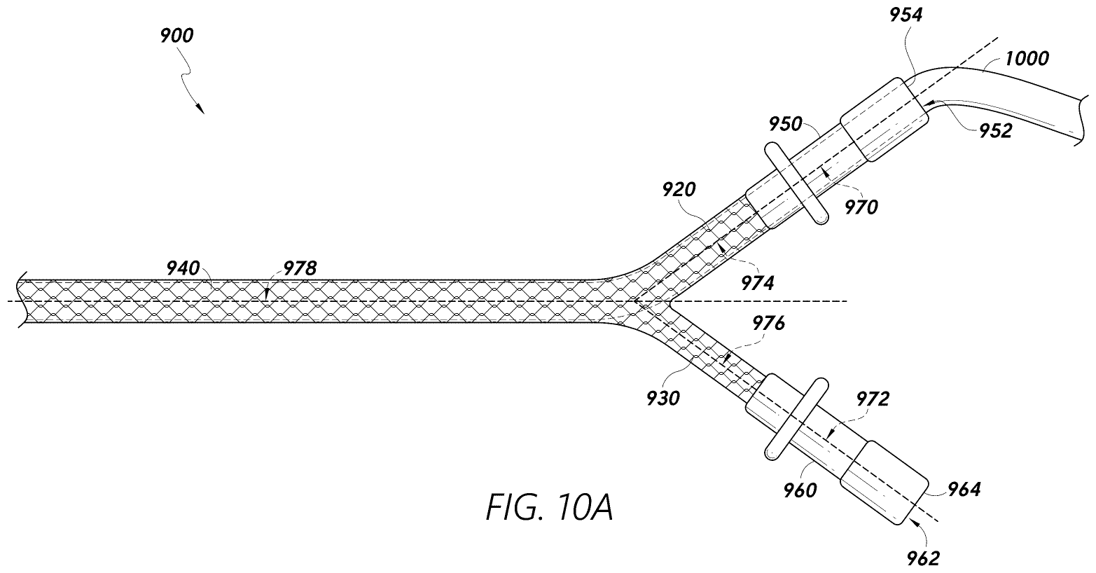

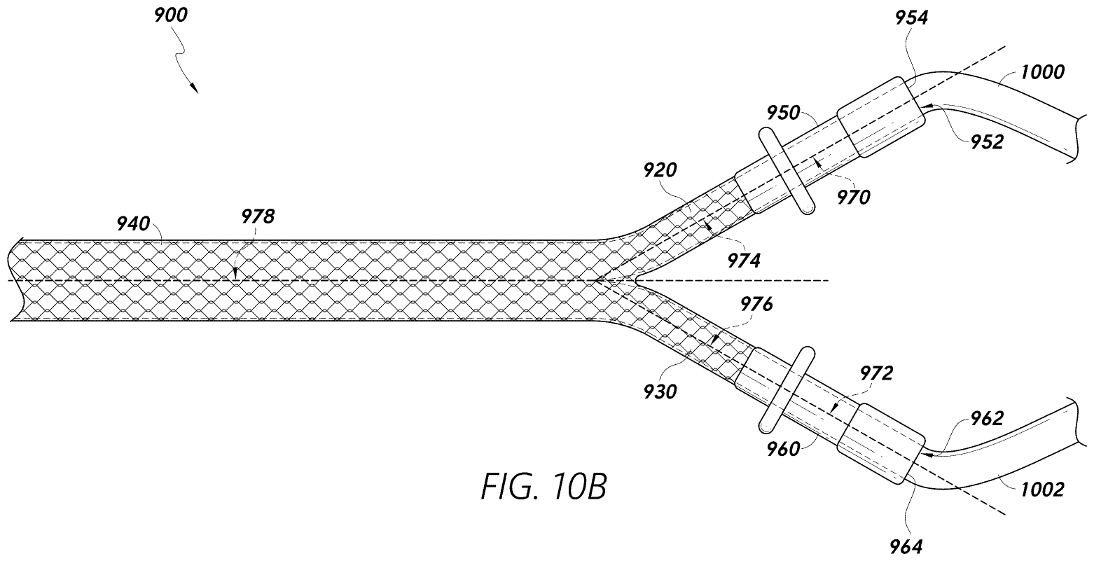

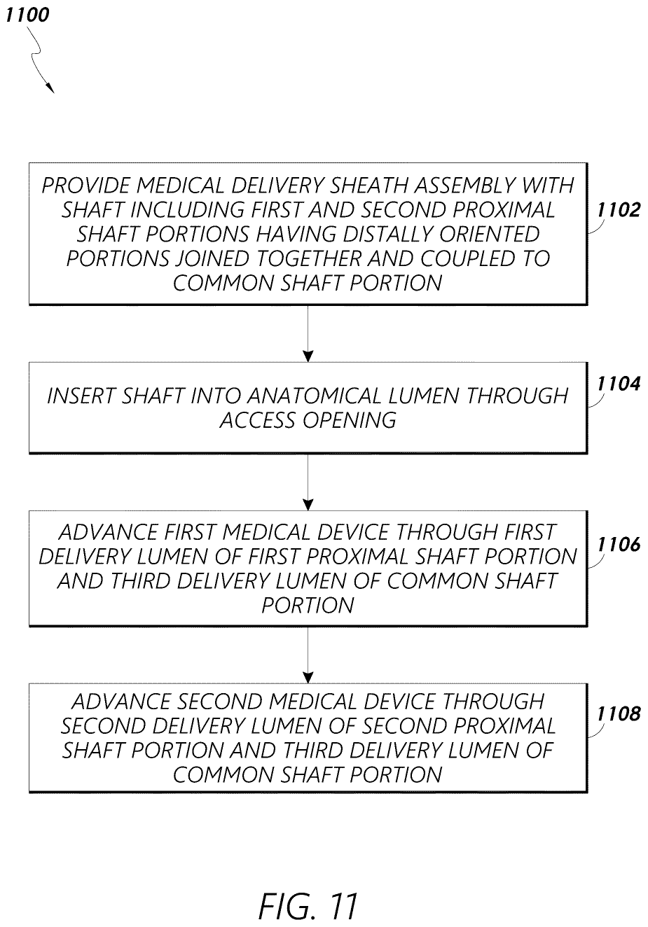

FIGS. 9A-10B show an example of a medical delivery sheath assembly 900 comprising a shaft 910 with a bifurcated proximal portion 912 to facilitate delivery of multiple medical devices. Branching in the shaft 910 can form the bifurcated proximal portion 912. For example, the shaft 910 can comprise a first proximal shaft portion 920 and a second proximal shaft portion 930. Distally oriented portions 924, 934 of the first and second proximal shaft portions 920, 930 can be joined to one another and coupled to a common shaft portion 940. Proximally oriented portions 922, 932 of the first and second proximal shaft portions 920, 930 can be coupled to a first hub member 950 and a second hub member 960, respectively. The first and second hub members 950, 960 can be configured to allow insertion therethrough of a respective medical device such that the medical delivery sheath assembly 900 can be used for advancement of multiple medical devices through a common insertion site. The shaft 910 can comprise a shape-memory material configured to facilitate reversible expansion of at least a portion of the shaft 910 to allow the shaft 910 to accommodate the medical devices. For example, the shaft 910 can comprise a bifurcated shape-memory material, including a bifurcated shape-memory layer, such as a bifurcated braided nitinol layer. The shape-memory layer can assume a bifurcated configuration, for example forming a branched shaft portion. The shape-memory layer can form at least a portion of a sidewall forming the bifurcation and/or branching in the shaft 910. FIG. 11 is a process flow diagram showing an example of a delivery process 1100 for delivery of multiple medical devices using a medical delivery sheath assembly, such as the medical delivery sheath assembly 900 described with reference to FIGS. 9A-10B.

Referring to FIG. 9A, a side view of the medical delivery sheath assembly 900 is shown. The medical delivery sheath assembly 900 can comprise the shaft 910 comprising the proximal shaft portion 912 that is bifurcated, and the first hub member 950 and the second hub member 960 configured to be coupled to the proximal portion 912 of the shaft. In some examples, the sheath assembly 900 can include two or more proximal shaft portions. The shaft 910 can comprise the first proximal shaft portion 920 and the second proximal shaft portion 930. The first proximally oriented portion 922 of the first proximal shaft portion 920 can be configured to be coupled to the first hub member 950. The second proximally oriented portion 932 of the second shaft proximal portion 930 can be configured to be coupled to the second hub member 960. The first distally oriented portion 924 of the first proximal shaft portion 920 and the second distally oriented portion 934 of the second proximal shaft portion 930 can be coupled to one another and to the common shaft portion 940. For example, a first distal end 928 of the first proximal shaft portion 920 and a second distal end 938 of the second proximal shaft portion 930 can be joined and/or merged together at and/or to form a joint 980. The common shaft portion 940 can extend distally from the joint 980. A proximal common shaft portion 942 can be associated with the joint 980, for example extending distally from the joint 980. For example, the common shaft portion 940 can extend from where the first proximal shaft portion 920 and the second proximal shaft portion 930 are joined to one another to a distal common shaft portion 944, including a distal end 918 of the shaft 910. The joint 980 may be an integral portion of the shaft 910, for example being integral with the first and second proximal shaft portion 920, 930 and the common shaft portion 940.

The first hub member 950 can comprise a first port, such as a first proximal port 952, configured to receive a first medical device. The first proximal port 952 can be on a proximal end 954 of the first hub member 950. The second hub member 960 can comprise a second port, such as a second proximal port 962, configured to receive a second medical device. The second proximal port 962 can be on a proximal end 964 of the second hub member 960. The first and second medical devices can be inserted into the medical delivery sheath assembly 900 for advancement into the vasculature through a common access site. For example, the first hub member 950 can comprise a first delivery lumen 970 extending through the first hub member 950. The first delivery lumen 970 can be in fluid communication with the first port, such as the first proximal port 952. The first delivery lumen 970 can be configured to slidably receive a corresponding portion of the first medical device. The second hub member 960 can comprise a second delivery lumen 972 extending through the second hub member 960. The second delivery lumen 972 can be in fluid communication with the second port, such as the second proximal port 962. The second delivery lumen 972 can be configured to slidably receive a corresponding portion of the second medical device. The first and second delivery lumens 970, 972 can extend along a first and second longitudinal axes, respectively. In some instances, the first and second longitudinal axes can extend along and/or be parallel or substantially parallel with the respective longitudinal axis of the first hub member 950 or the second hub member 960.

The first proximal shaft portion 920 can comprise a third delivery lumen 974 extending through the first proximal shaft portion 920 and configured to slidably receive a corresponding portion of the first medical device. The third delivery lumen 974 can be in fluid communication with the first delivery lumen 970. The second proximal shaft portion 930 can comprise a fourth delivery lumen 976 extending through the second proximal shaft portion 930 and configured to slidably receive a corresponding portion of the second medical device. The fourth delivery lumen 976 can be in fluid communication with the second delivery lumen 972. The third delivery lumen 974 and fourth delivery lumen 976 can extend along a third longitudinal axis and a fourth longitudinal axis, respectively. In some instances, the third and fourth longitudinal axes can extend along and/or be parallel or substantially parallel with the respective longitudinal axis of the first proximal shaft portion 920 or the second proximal shaft portion 930. A fifth delivery lumen 978 can extend along a fifth longitudinal axis through the common shaft portion 940. The fifth longitudinal axis can be coaxial and/or parallel or substantially parallel with the longitudinal axis of the common shaft portion 940. The fifth delivery lumen 978 can be in fluid communication with the third and fourth delivery lumens 974, 976. The fifth delivery lumen 978 can be configured to slidably receive corresponding portions of the first and second medical devices.

Corresponding portions of the first medical device can be advanced into the first delivery lumen 970 through the first proximal port 952 and from the first delivery lumen 970 into the third delivery lumen 974. The first medical device can be subsequently advanced into the fifth delivery lumen 978. Corresponding portions of the second medical device can be advanced into the second delivery lumen 972 through the second proximal port 962, from the second delivery lumen 972 into the fourth delivery lumen 976, and subsequently into the fifth delivery lumen 978. In some instances, portions of the first and second medical devices can be both slidably received by and be adjacent to one another within the fifth delivery lumen 978.

Referring again to FIG. 9A, in some instances, the third and the fourth longitudinal axes of the third and fourth delivery lumens 974, 976 can form an acute angle shown as “Θ₅” where the first and second proximal shaft portions 920, 930 are joined together. The acute angle “Os” formed by the third and fourth longitudinal axes does not cross the fifth longitudinal axis of the fifth delivery lumen 978. In some instances, the acute angle can be between about 10° and about 60°, including about 15° and about 45°. The angles formed by the third, fourth and fifth longitudinal axes can be selected to provide desired insertion of the medical devices. In some instances, the third longitudinal axis of the third delivery lumen 974 and the fourth longitudinal axis of the fourth delivery lumen 976 can be at a respective angle relative to the fifth longitudinal axis of the fifth delivery lumen 978, shown as “Θ₆” and “Θ₇”. The angle “Θ₆” formed by the third longitudinal axis and the fifth longitudinal axis can extend from the third longitudinal axis to the fifth longitudinal axis without crossing the fourth longitudinal axis. The angle “Θ₇” formed by the fourth longitudinal axis and the fifth longitudinal axis can extend from the fourth longitudinal axis to the fifth longitudinal axis without crossing the third longitudinal axis. In some instances, the angle “Θ₆” formed by the third longitudinal axis and the fifth longitudinal axis can be the same or similar to the angle “Θ₇” formed by the fourth longitudinal axis and the fifth longitudinal axis.

In some instances, the first proximal shaft portion 920, the second proximal shaft portion 930, and/or common shaft portion 940 can be flexibly joined together. For example, the joint 980 can comprise flexible, deformable and/or bendable material such that an orientation of the first proximal shaft portion 920, second proximal shaft portions 930, and/or common shaft portion 940 can change about the joint 980. The first and second proximal shaft portion 920, 930, and the common shaft portion 940 can form the angles “Θ₅”, “Θ₆” and/or “Θ₇” described herein while the shaft 910 in a relaxed state. The angles “Θ₅”, “Θ₆” and/or “Θ₇” can change while an operator is manipulating the medical delivery sheath assembly 900. In some instances, the orientation of the first proximal shaft portion 920, second proximal shaft portions 930, and/or common shaft portion 940 can change about the joint 980 to facilitate advancement of the medical devices.

FIG. 9B is a lateral cross-sectional view of the shaft 910 of the medical delivery sheath assembly 900. The lateral cross-sectional view is taken at a position on the common shaft portion 940 and along a plane perpendicular or substantially perpendicular to a longitudinal axis of the common shaft portion 940. The common shaft portion 940 can comprise a shape-memory layer 990, and one or more polymeric layers 992. In some instances, at least a portion of the shape-memory layer 990 can be between and/or sandwiched two polymeric layers 992. The common shaft portion 940 can comprise a polymeric layer 992 extending along and/or in contact with at least a portion of an internally oriented surface of the shape-memory layer 990 and another polymeric layer 992 over and/or in contact with at least a portion of an externally oriented surface of the shape memory layer 990. The shape-memory material can comprise nitinol. In some instances, the shape-memory layer 990 can comprise a braided shape memory material. For example, the shape-memory layer 990 can be a nitinol layer, including a braided nitinol layer. In some instances, one or both of the polymeric layers 992 can comprise polyethylene, including low density polyethylene (LDPE) and/or ultrahigh molecular weight polyethylene (UHMWPE). In some instances, one or both of the polymeric layers 992 can comprise both low density polyethylene (LDPE) and ultrahigh molecular weight polyethylene (UHMWPE). For example, one or both of the polymeric layers 992 can include one or more layers of low-density polyethylene (LDPE) and/or one or more layers of ultrahigh molecular weight polyethylene (UHMWPE). In some instances, each of the two polymeric layers 992 can include one or more layers of low-density polyethylene (LDPE) and/or one or more layers of ultrahigh molecular weight polyethylene (UHMWPE). In some instances, the two polymeric layers 992 can comprise the same material. For example, at least a portion of the shape-memory layer 990 can be between and/or sandwiched by two polymeric layers 992 with the same composition. Alternatively, at least a portion of the shape-memory layer 990 can be between and/or sandwiched by two polymer layers having different compositions.

In some instances, the first proximal shaft portion 920, the second proximal shaft portion 930 and the common shaft portion 940 can form an integral shaft. The first and second proximal shaft portions 920, 930 can join together and/or merge such that the common shaft portion 940 extends from where the first and second proximal shaft portions 920, 930 are joined and/or merged together. The integral shaft can comprise the shape-memory layer 990 and the polymeric layers 992 such that the shape-memory layer 990 and/or the polymeric layers 992 form at least the joint 980 of the shaft 910. The joint 980 can be an integral part of the shaft 910, for example formed at the position where the first and second proximal shaft portions 920, 930 merge. The shape-memory layer 990 and/or the polymeric layers 992 can each comprise a bifurcated portion. For example, the shaft 910 can comprise a bifurcated shape-memory layer portion, including a bifurcated nitinol layer portion, such as a bifurcated braided nitinol layer portion. The shape-memory layer 990 can have a bifurcated and/or branched shaft configuration. The shape-memory layer 990 can form at least a portion of a sidewall forming the bifurcation and/or branching in the shaft 910, for example such that the shape-memory layer 990 can have a shape of a bifurcated and/or branched shaft.

In some instances, each of the first and second proximal shaft portions 920, 930 and the common shaft portion 940 can comprise the shape-memory layer 990 and the polymeric layers 992. In some instances, the sidewall of the shaft 910 can be the shape-memory layer 990 and the polymeric layers 992. The shape-memory layer 990 can extend continuously along at least a portion of the shaft 910, including along at least a portion of the first proximal shaft portion 920, the second proximal shaft portion 930, the common shaft portion 940, and the joint 980. For example, a braided shape-memory layer, such as a braided nitinol layer, can extend continuously along at least a portion of the first proximal shaft portion 920, second proximal shaft portion 930, common shaft portion 940, and joint 980. The braided shape-memory layer can extend continuously along at least a portion of the first proximal shaft portion 920, second proximal shaft portion 930, common shaft portion 940, and joint 980, such that the braided shape-memory layer is a bifurcated and/or branched braided shape-memory layer. The braided shape-memory layer can assume a bifurcated and/or branched shaft shape.

In some instances, the distal common shaft portion 944 can comprise at least a portion that does not include the shape-memory layer 990. In some instances, a portion of the distal common shaft portion 944 can comprise a polymeric material. For example, the portion of the distal common shaft portion 944 can be polymeric. The polymeric material of the portion of the distal common shaft portion 944 may or may not be the same as one of the two polymeric layers 992. In some instances, the distal common shaft portion 944 can comprise a portion of one or both of the two polymeric layers 992 extending distally of a distal end of the shape-memory layer 990, such that the distal end 918 of the shaft 910 is a distal end of the distal end of the shape-memory layer 990.

As described herein, the shaft 910 can comprise at least a portion that is expandable. At least a portion of the shaft 910 can assume various expanded states to accommodate a first and a second medical device. FIG. 10A is a side view of the medical delivery sheath assembly 900 where the shaft 910 is in a first expanded state after a first medical device 1000 is advanced through the shaft 910. FIG. 10B is a side view of the medical delivery sheath assembly 900 where the shaft 910 is in a second expanded state after a second medical device 1002 is advanced through the shaft 910. In some instances, at least a portion of the first proximal shaft portion 920, second proximal shaft portion 930 and/or common shaft portion 940 can be expandable. In some instances, the first proximal shaft portion 920, second proximal shaft portion 930 and common shaft portion 940 can be expandable. For example, the shaft 910 can be an expandable shaft. The shaft 910 can assume a first expanded state while one medical device is disposed through the shaft 910, and a second expanded state while two medical devices are disposed through the shaft 910. The shaft 910 can be in a collapsed state while no medical devices are disposed through the shaft 910, such as to facilitate blood flow around the shaft 910.

FIG. 10A shows the first proximal shaft portion 920 in an expanded state and the common shaft portion 940 in a first expanded state. The first medical device 1000 can be disposed through the first proximal port 952 on the first hub member 950 and the first delivery lumen 970 of the first hub member 950. The first medical device 1000 can extend from the first delivery lumen 970 into the third delivery lumen 974 of the first proximal shaft portion 920 and then into the fifth delivery lumen 978 of the common shaft portion 940. The first proximal shaft portion 920 can expand to accommodate the first medical device 1000. In some instances, respective portions of the first proximal shaft portion 920 can expand to accommodate the first medical device 1000 as the first medical device 1000 is advanced through the first proximal shaft portion 920. In some instances, the first proximal shaft portion 920 can be in a collapsed state prior to insertion of the first medical device 1000. The first proximal shaft portion 920 can be configured to assume an expanded state while the first medical device 1000 is slidably received through the third delivery lumen 974.

FIG. 10B shows the second proximal shaft portion 930 in an expanded state and the common shaft portion 940 in a second expanded state. The second medical device 1002 can be disposed through the second proximal port 962 on the second hub member 960 and the second delivery lumen 972 of the second hub member 960. The second medical device 1002 can extend from the second delivery lumen 972 into the fourth delivery lumen 976 of the second proximal shaft portion 930 and then into the fifth delivery lumen 978 of the common shaft portion 940. The second proximal shaft portion 930 can expand to accommodate the second medical device 1002. In some instances, respective portions of the second proximal shaft portion 930 can expand to accommodate the second medical device 1002 as the second medical device 1002 is advanced through the second proximal shaft portion 930. In some instances, the second proximal shaft portion 930 can be in a collapsed state prior to insertion of the second medical device 1002. The second proximal shaft portion 930 can be configured to assume an expanded state while the second medical device 1002 is slidably received through the fourth delivery lumen 976.

The common shaft portion 940 can expand to accommodate both the first medical device 1000 and the second medical device 1002. In some instances, respective portions of the common shaft portion 940 can expand to accommodate the second medical device 1002 as the second medical device 1002 is advanced through the common shaft portion 940. The common shaft portion 940 can expand from the first expanded state (with just the first medical device 1000) to the second expanded state (with both the first medical device 1000 and the second medical device 1002). The common expandable shaft portion 940 can be configured to assume the second expanded state while both first and second medical devices are slidably received within the fifth delivery lumen 978. Portions of the first and second medical devices 1000, 1002 can be slidably within the fifth delivery lumen 978 adjacent to and in contact with one another. It will be understood that the order of insertion of the medical devices is for illustrative purposes. For example, the second medical device 1002 can be inserted prior to insertion of the first medical device 1000.

FIG. 11 is a process flow diagram illustrating an example of a delivery process 1100 for delivering multiple medical devices, including using the medical delivery sheath assembly 900 described with reference to FIGS. 9A-10B. In block 1102, the process can involve providing a medical delivery sheath assembly. The medical delivery sheath assembly can have a shaft comprising a first proximal shaft portion with a first delivery lumen extending through the first proximal shaft portion and a second proximal shaft portion with a second delivery lumen extending through the second proximal shaft portion. Distally oriented portions of the first and second proximal shaft portions can be joined together and coupled to a common shaft portion. The common shaft portion can comprise a third delivery lumen extending through the common shaft portion and in fluid communication with the first and second delivery lumens. In some instances, the common shaft portion can extend distally from the first and second proximal shaft portions to a distal end of the shaft.

In block 1104, the process can involve inserting at least a portion of the shaft into an anatomical lumen through an access opening. The access opening can be formed on any number of anatomical lumens and/or vessels. In some instances, the anatomical lumen can be a blood vessel, such as a femoral artery. For example, inserting at least a portion of the shaft into the anatomical lumen through the access opening can comprise inserting at least a portion of the shaft into an access opening formed on a femoral artery, including a left or a right femoral artery. In some instances, a venous access site can be used, including a femoral, jugular or sub-clavian venous access site.

In block 1106, the process can involve, advancing at least a portion of a first medical device through the first delivery lumen of first proximal shaft portion and the third delivery lumen of common shaft portion. In block 1108, the process can involve, advancing at least a portion of a second medical device through the second delivery lumen of the second proximal shaft portion and the third delivery lumen of the common shaft portion.

In some instances, advancing at least a portion of the first medical device into the first delivery lumen can comprise expanding the first proximal shaft portion from a collapsed state to an expanded state. In some instances, advancing at least a portion of the second medical device into the second delivery lumen can comprise expanding the second proximal shaft portion from a collapsed state to an expanded state. In some instances, advancing at least a portion of the first medical device into the third delivery lumen can comprise expanding the common shaft portion from a collapsed state to a first expanded state. In some instances, advancing at least a portion of the second medical device into the third delivery lumen can comprise expanding the common shaft portion from the first expanded state to a second expanded state. In some instances, advancing at least a portion of the second medical device into the third delivery lumen can comprise advancing the at least a portion of the second medical device into the third delivery lumen alongside and adjacent to the at least a portion of the first medical device slidably received in the third delivery lumen.

In some instances, advancing at least a portion of the first medical device through the first delivery lumen and the third delivery lumen can comprise advancing at least a portion of a mechanical circulatory support (MCS) device through the first delivery lumen and the third delivery lumen. In some instances, advancing at least a portion of a second medical device through the second delivery lumen and the third delivery lumen can comprise advancing a percutaneous coronary intervention (PCI) device through the first delivery lumen and the third delivery lumen. In some instances, the process can involve advancing at least a portion of the mechanical circulatory support (MCS) device into a left heart. In some instances, the process can involve advancing at least a portion of the percutaneous coronary intervention (PCI) device into a coronary artery.

In some instances, a longitudinal axis of the first proximal shaft portion and a longitudinal axis of the second proximal shaft portion can each be at a respective angle relative to a longitudinal axis of the common shaft portion. For example, advancing at least a portion of the first medical device through the first delivery lumen can comprise advancing the at least a portion of the first medical device through a first delivery lumen comprising a longitudinal axis at a first angle relative to a longitudinal axis of the third delivery lumen. In some instances, advancing at least a portion of the second medical device through the second delivery lumen can comprise advancing the at least a portion of the second medical device through a second delivery lumen comprising a longitudinal axis at a second angle relative to the longitudinal axis of the third delivery lumen.

### Additional Description of Examples

Provided below is a list of examples, each of which may include aspects of any of the other examples disclosed herein. Furthermore, aspects of any example described above may be implemented in any of the numbered examples provided below.

Example 1: A medical delivery sheath assembly comprising a first hub portion comprising a first delivery lumen extending therethrough and configured to slidably receive at least a portion of a first medical device, and a second hub portion comprising a second delivery lumen extending therethrough and configured to slidably receive at least a portion of a second medical device. A first longitudinal axis of the first delivery lumen and a second longitudinal axis of the second delivery lumen can form an acute angle where a distal portion of the first hub portion merges with a distal portion of the second hub portion. The assembly can include a joint hub portion disposed distally of and coupled to the distal portion of the first hub portion and the distal portion of the second hub portion, the joint hub portion comprising a third delivery lumen extending therethrough and configured to slidably receive corresponding portions of each the first medical device and the second medical device.

Example 2: The assembly of any example herein, in particular example 1, further comprising a shaft, the shaft extending distally from a distal end of the joint hub portion and comprising a shaft delivery lumen extending therethrough and being configured to slidably receive corresponding portions of each the first medical device and the second medical device.

Example 3: The assembly of any example herein, in particular example 2, wherein the shaft comprises an expandable shaft portion, and wherein the expandable shaft portion is configured to assume a first expanded state while at least a portion of the first medical device is slidably received within the shaft delivery lumen, and assume a second expanded state while at least a portion of the first medical device and at least a portion of the second medical device are slidably received within the shaft delivery lumen.

Example 4: The assembly of any example herein, in particular example 3, wherein a lateral cross-section of the expandable shaft portion comprises an oval shape in the second expanded state.

Example 5: The assembly of any example herein, in particular example 3 or 4, wherein at least a portion of the first medical device and at least a portion of the second medical device are configured to be slidably received within a single shaft delivery lumen.

Example 6: The assembly of any example herein, in particular examples 1 to 5, wherein the acute angle is between 15° and 45°.

Example 7: The assembly of any example herein, in particular examples 1 to 6, further comprising a flush port on the first hub portion.

Example 8: The assembly of any example herein, in particular examples 1 to 7, further comprising a flush port on the second hub portion.

Example 9: The assembly of any example herein, in particular examples 1 to 8, further comprising a flush port on the joint hub portion.

Example 10: The assembly of any example herein, in particular examples 1 to 9, wherein the first delivery lumen is configured to slidably receive at least a portion of a mechanical circulatory support (MCS) device.

Example 11: The assembly of any example herein, in particular examples 1 to 10, wherein the second delivery lumen is configured to slidably receive at least a portion of a percutaneous coronary intervention (PCI) device.

Example 12: The assembly of any example herein, in particular examples 1 to 11, wherein the longitudinal axis of the first delivery lumen is coaxial with a longitudinal axis of the shaft delivery lumen, and the longitudinal axis of the second delivery lumen is at an angle relative to the longitudinal axis of the shaft delivery lumen.

Example 13: The assembly of any example herein, in particular examples 1 to 11, wherein the longitudinal axis of the first delivery lumen and the longitudinal axis of the second delivery lumen are at a respective angle relative to a longitudinal axis of the shaft delivery lumen.

Example 14: A method of delivering a medical device, the method comprising providing a medical delivery sheath assembly. The medical delivery sheath assembly can comprise a hub member having a first hub portion that includes a first delivery lumen and a second hub portion that includes a second delivery lumen, a first longitudinal axis of the first delivery lumen and a second longitudinal axis of the second delivery lumen forming an acute angle where a distal portion of the first hub portion merges with a distal portion of the second hub portion. The medical delivery sheath assembly can include a joint hub portion distally disposed from and coupled to the distal portion of the first hub portion and the distal portion of the second hub portion and comprising a third delivery lumen, and a shaft extending distally from a distal end of the joint hub portion and comprising a shaft delivery lumen. The method can include inserting at least a portion of the shaft into an anatomical lumen through an access opening, advancing at least a portion of a first medical device through the first delivery lumen and the third delivery lumen, and into the shaft delivery lumen, and advancing at least a portion of a second medical device through the second delivery lumen and the third delivery lumen, and into the shaft delivery lumen.

Example 15: The method of any example herein, in particular example 14, wherein advancing at least a portion of the first medical device into the shaft delivery lumen comprises advancing the at least a portion of the medical device into a corresponding portion of the shaft delivery lumen extending through an expandable shaft portion and expanding the expandable shaft portion from a collapsed state to a first expanded state.

Example 16: The method of any example herein, in particular example 15, wherein advancing at least a portion of the second medical device into the shaft delivery lumen comprises advancing the at least a portion of the second medical device into the corresponding portion of the shaft delivery lumen extending through the expandable shaft portion and expanding the expandable shaft portion from the first expanded state to a second expanded state.

Example 17: The method of any example herein, in particular examples 14 to 16, wherein advancing at least a portion of the second medical device into the shaft delivery lumen comprises advancing the at least a portion of the second medical device into the shaft delivery lumen alongside and adjacent to the at least a portion of the first medical device slidably received in the shaft delivery lumen.

Example 18: The method of any example herein, in particular examples 14 to 17, wherein advancing at least a portion of the first medical device through the first delivery lumen and the third delivery lumen, and into the shaft delivery lumen comprises advancing at least a portion of a mechanical circulatory support (MCS) device through the first delivery lumen and the third delivery lumen, and into the shaft delivery lumen, and advancing at least a portion of a second medical device through the second delivery lumen and the third delivery lumen, and into the shaft delivery lumen comprises advancing a percutaneous coronary intervention (PCI) device through the first delivery lumen and the third delivery lumen, and into the shaft delivery lumen.

Example 19: The method of any example herein, in particular example 18, further comprising advancing at least a portion of the mechanical circulatory support (MCS) device into a left heart, and advancing at least a portion of the percutaneous coronary intervention (PCI) device into a coronary artery.

Example 20: The method of any example herein, in particular examples 14 to 19, wherein inserting at least a portion of the shaft into the anatomical lumen through the access opening comprises inserting at least a portion of the shaft into an access opening formed on a femoral artery.

Example 21: The method of any example herein, in particular examples 14 to 20, wherein advancing at least a portion of the first medical device through the first delivery lumen comprises advancing the at least a portion of the first medical device through a first delivery lumen comprising a longitudinal axis coaxial to a longitudinal axis of the shaft delivery lumen, and advancing at least a portion of the second medical device through the second delivery lumen comprises advancing the at least a portion of the second medical device through a second delivery lumen comprising a longitudinal axis at an angle relative to the longitudinal axis of the shaft delivery lumen.

Example 22: The method any example herein, in particular examples 14 to 20, wherein advancing at least a portion of the first medical device through the first delivery lumen comprises advancing the at least a portion of the first medical device through a first delivery lumen comprising a longitudinal axis at a first angle relative to a longitudinal axis of the shaft delivery lumen, and advancing at least a portion of the second medical device through the second delivery lumen comprises advancing the at least a portion of the second medical device through a second delivery lumen comprising a longitudinal axis at a second angle relative to the longitudinal axis of the shaft delivery lumen.

The above method(s) can be performed on a living animal or on a simulation, such as on a cadaver, cadaver heart, anthropomorphic ghost, simulator (e.g., with body parts, heart, tissue, etc. being simulated).

Example 23: A medical delivery sheath assembly comprising a first hub member comprising a first proximal port configured to receive at least a portion of a first medical device and a second hub member comprising a second proximal port configured to receive at least a portion of a second medical device. The medical delivery sheath assembly can include a shaft comprising a first proximal shaft portion and a second proximal shaft portion, a proximally oriented portion of the first proximal shaft portion being coupled to the first hub member, a proximally oriented portion of the second shaft proximal portion being coupled to the second hub member, and distally oriented portions of the first and second proximal shaft portions being joined together and coupled to a common shaft portion.

Example 24: The assembly of any example herein, in particular example 23, wherein the first proximal shaft portion and the second proximal shaft portion and the common shaft portion form an integral shaft, the first and second proximal shaft portions joining together to form the common shaft portion.

Example 25: The assembly of any example herein, in particular example 23 or 24, wherein the common shaft portion extends from the first proximal shaft portion and the second proximal shaft portion to a distal end of the shaft.

Example 26: The assembly of any example herein, in particular examples 23 to 25, wherein the first proximal shaft portion and the second proximal shaft portion are flexibly joined together.

Example 27: The assembly of any example herein, in particular examples 23 to 26, wherein the shaft comprises a shape memory layer.

Example 28: The assembly of any example herein, in particular example 27, wherein the shaft comprises the shape-memory layer between two polymeric layers.

Example 29: The assembly of any example herein, in particular example 27 or 28, wherein the shape-memory layer comprises a braided shape-memory material.

Example 30: The assembly of any example herein, in particular examples 23 to 29, wherein a first delivery lumen in fluid communication with the first proximal port extends through the first hub member and is configured to slidably receive a corresponding portion of the first medical device, a second delivery lumen in fluid communication with the second proximal port extends through the second hub member and is configured to slidably receive a corresponding portion of the second medical device, a third delivery lumen extending along a third longitudinal axis through the first proximal shaft portion is in fluid communication with the first delivery lumen, and is configured to slidably receive a corresponding portion of the first medical device, and a fourth delivery lumen extending along a fourth longitudinal axis through the second proximal shaft portion is in fluid communication with the second delivery lumen, and is configured to slidably receive a corresponding portion of the second medical device. The third and the fourth longitudinal axes can form an acute angle where the first and second proximal shaft portions join together.

Example 31: The assembly of any example herein, in particular example 30, wherein a fifth delivery lumen extending along a fifth longitudinal axis through the common shaft portion is in fluid communication with the third and fourth delivery lumens, the fifth delivery lumen being configured to slidably receive corresponding portions of the first and second medical devices.

Example 32: The assembly of any example herein, in particular example 31, wherein the third longitudinal axis of the third delivery lumen and the fourth longitudinal axis of the fourth delivery lumen are at a respective angle relative to the fifth longitudinal axis of the common shaft portion.

Example 33: The assembly of any example herein, in particular examples 30 to 32, wherein the first proximal shaft portion comprises a first proximal expandable shaft portion, and wherein the first proximal expandable shaft portion is configured to assume an expanded state while at least a portion of the first medical device is slidably received within the third delivery lumen, and the second proximal shaft portion comprises a second proximal expandable shaft portion, and wherein the second proximal expandable shaft portion is configured to assume an expanded state while at least a portion of the second medical device is slidably received within the fourth delivery lumen.

Example 34: The assembly of any example herein, in particular examples 31 to 33, wherein the common shaft comprises a common expandable shaft portion, and wherein the common expandable shaft portion is configured to assume a first expanded state while at least a portion of the first medical device is slidably received within the fifth delivery lumen, and assume a second expanded state while at least a portion of the first medical device and at least a portion of the second medical device are slidably received within the fifth delivery lumen.

Example 35: A method of delivering a medical device, the method comprising providing a medical delivery sheath assembly. The medical delivery sheath assembly can have a shaft comprising a first proximal shaft portion comprising a first delivery lumen extending therethrough and a second proximal shaft portion comprising a second delivery lumen extending therethrough. Distally oriented portions of the first and second proximal shaft portions can be joined together and coupled to a common shaft portion. The common shaft portion can comprise a third delivery lumen extending therethrough and in fluid communication with the first and second delivery lumens. The method can include inserting at least a portion of the shaft into an anatomical lumen through an access opening, advancing at least a portion of a first medical device through the first delivery lumen of the first proximal shaft portion and the third delivery lumen of the common shaft portion, and advancing at least a portion of a second medical device through the second delivery lumen of the second proximal shaft portion and the third delivery lumen of the common shaft portion.

Example 36: The method of any example herein, in particular example 35, wherein advancing at least a portion of the first medical device into the first delivery lumen and the third delivery lumen comprises expanding the first proximal shaft portion from a collapsed state to an expanded state and expanding the common shaft portion from a collapsed state to a first expanded state.

Example 37: The method of any example herein, in particular example 36, wherein advancing at least a portion of the second medical device into the second delivery lumen and the third delivery lumen comprises expanding the second proximal shaft portion from a collapsed state to an expanded state and expanding the common shaft portion from the first expanded state to a second expanded state.

Example 38: The method of any example herein, in particular example 37, wherein advancing at least a portion of the second medical device into the third delivery lumen comprises advancing the at least a portion of the second medical device into the third delivery lumen alongside and adjacent to the at least a portion of the first medical device slidably received in the third delivery lumen.

Example 39: The method of any example herein, in particular examples 35 to 38, wherein advancing at least a portion of the first medical device through the first delivery lumen and the third delivery lumen comprises advancing at least a portion of a mechanical circulatory support (MCS) device through the first delivery lumen and the third delivery lumen, and advancing at least a portion of a second medical device through the second delivery lumen and the third delivery lumen comprises advancing a percutaneous coronary intervention (PCI) device through the first delivery lumen and the third delivery lumen.

Example 40: The method of any example herein, in particular example 39, further comprising advancing at least a portion of the mechanical circulatory support (MCS) device into a left heart, and advancing at least a portion of the percutaneous coronary intervention (PCI) device into a coronary artery.

Example 41: The method of any example herein, in particular examples 35 to 40, wherein inserting at least a portion of the shaft into the anatomical lumen through the access opening comprises inserting at least a portion of the shaft into an access opening formed on a femoral artery.

Example 42: The method of any example herein, in particular examples 35 to 41, wherein advancing at least a portion of the first medical device through the first delivery lumen comprises advancing the at least a portion of the first medical device through a first delivery lumen comprising a longitudinal axis at a first angle relative to a longitudinal axis of the third delivery lumen, and advancing at least a portion of the second medical device through the second delivery lumen comprises advancing the at least a portion of the second medical device through a second delivery lumen comprising a longitudinal axis at a second angle relative to the longitudinal axis of the third delivery lumen.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical delivery sheath assembly comprising:
a first hub portion comprising a first delivery lumen extending therethrough and configured to slidably receive at least a portion of a first medical device;
a second hub portion comprising a second delivery lumen extending therethrough and configured to slidably receive at least a portion of a second medical device, a first longitudinal axis of the first delivery lumen and a second longitudinal axis of the second delivery lumen forming an acute angle where a distal portion of the first hub portion merges with a distal portion of the second hub portion; and
a joint hub portion disposed distally of and coupled to the distal portion of the first hub portion and the distal portion of the second hub portion, the joint hub portion comprising a third delivery lumen extending therethrough and configured to slidably receive corresponding portions of each the first medical device and the second medical device.

2. The assembly of claim 1, further comprising a shaft, the shaft extending distally from a distal end of the joint hub portion and comprising a shaft delivery lumen extending therethrough and being configured to slidably receive corresponding portions of each the first medical device and the second medical device.

3. The assembly of claim 2, wherein the shaft comprises an expandable shaft portion, and wherein the expandable shaft portion is configured to:
assume a first expanded state while at least a portion of the first medical device is slidably received within the shaft delivery lumen; and
assume a second expanded state while at least a portion of the first medical device and at least a portion of the second medical device are slidably received within the shaft delivery lumen.

4. The assembly of claim 3, wherein a lateral cross-section of the expandable shaft portion comprises an oval shape in the second expanded state.

5. The assembly of claim 3, wherein at least a portion of the first medical device and at least a portion of the second medical device are configured to be slidably received within a single shaft delivery lumen.

6. The assembly of claim 2, wherein the longitudinal axis of the first delivery lumen is coaxial with a longitudinal axis of the shaft delivery lumen, and the longitudinal axis of the second delivery lumen is at an angle relative to the longitudinal axis of the shaft delivery lumen.

7. The assembly of claim 2, wherein the longitudinal axis of the first delivery lumen and the longitudinal axis of the second delivery lumen are at a respective angle relative to a longitudinal axis of the shaft delivery lumen.

8. The assembly of claim 1, further comprising a flush port on the first hub portion, second hub portion or joint hub portion.

9. The assembly of claim 1, wherein the first delivery lumen is configured to slidably receive at least a portion of a mechanical circulatory support (MCS) device, and wherein the second delivery lumen is configured to slidably receive at least a portion of a percutaneous coronary intervention (PCI) device.

10. A medical delivery sheath assembly comprising:
a first hub member comprising a first proximal port configured to receive at least a portion of a first medical device;
a second hub member comprising a second proximal port configured to receive at least a portion of a second medical device; and
a shaft comprising a first proximal shaft portion and a second proximal shaft portion, a proximally oriented portion of the first proximal portion being coupled to the first hub member, a proximally oriented portion of the second shaft proximal portion being coupled to the second hub member, and distally oriented portions of the first and second proximal shaft portions being joined together and coupled to a common shaft portion.

11. The assembly of claim 10, wherein the first proximal shaft portion and the second proximal shaft portion and the common shaft portion form an integral shaft, the first and second proximal shaft portions joining together to form the common shaft portion.

12. The assembly of claim 10, wherein the common shaft portion extends from the first proximal shaft portion and the second proximal shaft portion to a distal end of the shaft.

13. The assembly of claim 10, wherein the first proximal shaft portion and the second proximal shaft portion are flexibly joined together.

14. The assembly of claim 10, wherein the shaft comprises a shape-memory layer between two polymeric layers.

15. The assembly of claim 14, wherein the shape-memory layer comprises a braided shape-memory material.

16. The assembly of claim 10, wherein:
a first delivery lumen is in fluid communication with the first proximal port extends through the first hub member and is configured to slidably receive a corresponding portion of the first medical device;
a second delivery lumen is in fluid communication with the second proximal port extends through the second hub member and is configured to slidably receive a corresponding portion of the second medical device;
a third delivery lumen extending along a third longitudinal axis is in fluid communication with the first delivery lumen and extends through the first proximal shaft portion, and is configured to slidably receive a corresponding portion of the first medical device; and
a fourth delivery lumen extending along a fourth longitudinal axis is in fluid communication with the second delivery lumen and extends through the second proximal shaft portion, and is configured to slidably receive a corresponding portion of the second medical device, the third and the fourth longitudinal axes forming an acute angle where the first and second proximal shaft portions join together.

17. The assembly of claim 16, wherein a fifth delivery lumen extending along a fifth longitudinal axis through the common shaft portion is in fluid communication with the third and fourth delivery lumens, the fifth delivery lumen being configured to slidably receive corresponding portions of the first and second medical devices.

18. The assembly of claim 17, wherein the third longitudinal axis of the third delivery lumen and the fourth longitudinal axis of the fourth delivery lumen are at a respective angle relative to the fifth longitudinal axis of the common shaft portion.

19. The assembly of claim 17, wherein the common shaft comprises a common expandable shaft portion, and wherein the common expandable shaft portion is configured to:
assume a first expanded state while at least a portion of the first medical device is slidably received within the fifth delivery lumen; and
assume a second expanded state while at least a portion of the first medical device and at least a portion of the second medical device are slidably received within the fifth delivery lumen.

20. The assembly of claim 10, wherein:
the first proximal shaft portion comprises a first proximal expandable shaft portion, and wherein the first proximal expandable shaft portion is configured to assume an expanded state while at least a portion of the first medical device is slidably received within the third delivery lumen; and
the second proximal shaft portion comprises a second proximal expandable shaft portion, and wherein the second proximal expandable shaft portion is configured to assume an expanded state while at least a portion of the second medical device is slidably received within the fourth delivery lumen.
